SEQUENCE LISTING (14 sequences)

SEQ ID NO: 1 - protein, 614 aa, Mucor circinelloides
MLLTYMEVHLYYTLPVLGVLSWLSRPYYTATDALKFKFLTLVAFTTASAWDNYIVYHKAWSYCPTCVTAVIGYVPLEEYMFFIIMTLLTVAFTNLVMRWHLHSFFIRPETPVMQSVLVRLVPITALLITAYKAWHLAVPGKPLFYGSCILWYACPVLALLWFGAGEYMMRRPLAVLVSIALPTLFLCWVDVVAIGAGTWDISLATSTGKFVVPHLPVEEFMFFALINTVLVFGTCAIDRTMAILHLFKNKSPYQRPYQHSKSFLHQILEMTWAFCLPDQVLHSDTFHDLSVSWDILRKASKSFYTASAVFPGDVRQELGVLYAFCRATDDLCDNEQVPVQTRKEQLILTHQFVSDLFGQKTSAPTAIDWDFYNDQLPASCISAFKSFTRLRHVLEAGAIKELLDGYKWDLERRSIRDQEDLRYYSACVASSVGEMCTRIILAHADKPASRQQTQWIIQRAREMGLVLQYTNIARDIVTDSEELGRCYLPQDWLTEKEVALIQGGLAREIGEERLLSLSHRLIYQADELMVVANKGIDKLPSHCQGGVRAACNVYASIGTKLKSYKHHYPSRAHVGNSKRVEIALLSVYNLYTAPIATSSTTHCRQGKMRNLNTI

SEQ ID NO: 2 - DNA, 1845 bp, Mucor circinelloides
  source 1..1845: mol_type = genomic DNA
atgctcctgacctacatggaggtccacctctactacaccctgcccgttctcggtgttctgtcttggctctcccgaccttactacaccgccaccgacgctctcaagttcaagttcctgaccctcgttgcctttaccactgcctccgcctgggacaactacatcgtctaccacaaggcctggtcctactgccccacctgtgtcaccgccgtcattggttacgttcctctcgaggagtacatgttctttatcatcatgaccctgctcaccgtcgccttcaccaacctcgtcatgcgatggcacctccactccttcttcatccgacccgagactcccgtcatgcagtctgttctcgtccgacttgtccccatcactgctctgctcatcaccgcctacaaggcttggcaccttgctgtccccggcaagcccctcttctacggctcttgtatcctgtggtacgcttgccccgtcctcgctctcctctggtttggcgccggcgagtacatgatgcgacgacccctggccgttctcgtctccattgccctgcccactctcttcctgtgctgggtcgatgttgtcgccattggtgccggtacctgggacatctctctggccacttccactggtaagttcgttgtcccccacctccccgtcgaggagttcatgttcttcgccctcatcaacaccgtcctcgtctttggtacctgtgccatcgaccgaaccatggccatcctccacctcttcaagaacaagtctccttaccagcgaccctaccagcactccaagtccttcctgcaccagattctcgagatgacctgggccttctgtctgcccgaccaggttctgcactctgacaccttccacgacctctccgtctcttgggatatcctccgaaaggcctctaagtccttctacaccgcttccgctgtcttccccggtgacgtccgacaggagcttggtgttctctacgccttctgccgagccaccgatgacctctgtgacaacgagcaggtccccgtccagacccgaaaggagcagctcatcctcacccaccagtttgtctccgacctctttggccagaagacctccgcccccaccgccattgactgggacttctacaacgaccagctccccgcttcctgcatctccgccttcaagtctttcacccgactccgacacgttctcgaggctggtgccatcaaggagctgctcgacggttacaagtgggatctcgagcgacgatccatccgagatcaagaggacctccgatactactccgcctgtgttgcctcttccgtcggtgagatgtgcacccgaatcatcctcgcccacgctgacaagcccgcctcccgacagcagactcagtggatcatccagcgagcccgagagatgggcctcgttctccagtacaccaacattgcccgagacattgtcactgactccgaggagctcggccgatgctacctcccccaggactggctcaccgagaaagaggttgctctcatccagggcggtcttgctcgagagattggtgaggagcgactcctctctctgtcccaccgactcatctaccaggctgacgagctcatggtcgtcgccaacaagggtatcgacaagctcccctcccactgccagggcggcgtccgagctgcctgcaacgtctacgcctccatcggtaccaagctcaagtcctacaagcaccactacccctctcgagcccacgtcggtaactccaagcgagtcgagattgctctgctgtccgtctacaacctgtacaccgcccccattgccacctcctccaccacccactgccgacagggcaagatgcgaaacctcaacacgatctaa

SEQ ID NO: 3 - DNA, 10291 bp, synthetic construct
  source 1..10291: mol_type = other DNA
ccaatgatgcggagacctggccatattggccagtggtgggccaggtgagaaaaaaaggtgtagtagatgttagagggaggggttgccgtacaagagttcggagaacatatgctaattgcactgtgcttgtaagtggatgacagaagtgccagagacggaggtattatttcattgaaagacagacggcagctagcacgcaccgtccacaagcagtatcattcaggtctgggatgtgtcttgacctggacattacacttgggctgcacacgggacaaaacttagttgagttagagtctatgtgcagttcggtatgtactgcccagtattatagcagtcgacacagcactcaccaaacctgctacgaaccagtcaacccaactccaccacacagaaacaacactcagaaacacacacaccacgttccacatgtcgtctctgaaccccaaccgtgcaaacaaggtcgacgtgtcgtctctgtcacctgaagagcagcggattttcaagctctacggcaagctgcctacccagaaggacgtgctttccaagaaaatcaacgagcgaaagtacttcgactctggagattatgccatgagccgggctggacgggtcgactcggtagatgccggccaggtcggacttcagcaccccaaccccgaaaagatctctaagctccagcacaacggttgtggaggaggttcgtgtgctcctggcggctcgtcgcccccaggtgtccagtcgcctatcaaagagagcagccagctgagccagaacaactaggggacagcaggagaacggagctaagacgagcggatatgacgatgaacgatacggcgtctcgcggcagagcccgcaaaactaacacaaacgccatggcctgtgtgagcccactatcgcgcacaataagacgagagtacaccacacgccactaacggtccaacaaggagaattaagtatgtgaatgagtgaatgatattagatatgttgtagggtatagatatacttgtactgtgtagagttctccagcgcaacgtctgtgcgatagaaatggagatgtcgatgctgacctataaaagatgcaagtatgtcagtcaattgatatgtaaaagtaacagggtccctggtatacttgtagatttatcagcacaattttatcaacacaagctagacagtgaaatttttcactgcctttgctaacttcaaggggaactatagtaacatctttcacgagcgcttgtttatgctggtaacaaaaccgtccaagggaaactacaagtaaaagtagctacatgtagtctaagtcactctacaattagataatgatatatgatctgcatatctatgtcgggtgcggagaaagaggtaatgaaatggataagcttggaacaggatatttcgatcttggatacgtactcgcttgtgtctggtttcgtacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacaatgctcctgacctacatggaggtccacctctactacaccctgcccgttctcggtgttctgtcttggctctcccgaccttactacaccgccaccgacgctctcaagttcaagttcctgaccctcgttgcctttaccactgcctccgcctgggacaactacatcgtctaccacaaggcctggtcctactgccccacctgtgtcaccgccgtcattggttacgttcctctcgaggagtacatgttctttatcatcatgaccctgctcaccgtcgccttcaccaacctcgtcatgcgatggcacctccactccttcttcatccgacccgagactcccgtcatgcagtctgttctcgtccgacttgtccccatcactgctctgctcatcaccgcctacaaggcttggcaccttgctgtccccggcaagcccctcttctacggctcttgtatcctgtggtacgcttgccccgtcctcgctctcctctggtttggcgccggcgagtacatgatgcgacgacccctggccgttctcgtctccattgccctgcccactctcttcctgtgctgggtcgatgttgtcgccattggtgccggtacctgggacatctctctggccacttccactggtaagttcgttgtcccccacctccccgtcgaggagttcatgttcttcgccctcatcaacaccgtcctcgtctttggtacctgtgccatcgaccgaaccatggccatcctccacctcttcaagaacaagtctccttaccagcgaccctaccagcactccaagtccttcctgcaccagattctcgagatgacctgggccttctgtctgcccgaccaggttctgcactctgacaccttccacgacctctccgtctcttgggatatcctccgaaaggcctctaagtccttctacaccgcttccgctgtcttccccggtgacgtccgacaggagcttggtgttctctacgccttctgccgagccaccgatgacctctgtgacaacgagcaggtccccgtccagacccgaaaggagcagctcatcctcacccaccagtttgtctccgacctctttggccagaagacctccgcccccaccgccattgactgggacttctacaacgaccagctccccgcttcctgcatctccgccttcaagtctttcacccgactccgacacgttctcgaggctggtgccatcaaggagctgctcgacggttacaagtgggatctcgagcgacgatccatccgagatcaagaggacctccgatactactccgcctgtgttgcctcttccgtcggtgagatgtgcacccgaatcatcctcgcccacgctgacaagcccgcctcccgacagcagactcagtggatcatccagcgagcccgagagatgggcctcgttctccagtacaccaacattgcccgagacattgtcactgactccgaggagctcggccgatgctacctcccccaggactggctcaccgagaaagaggttgctctcatccagggcggtcttgctcgagagattggtgaggagcgactcctctctctgtcccaccgactcatctaccaggctgacgagctcatggtcgtcgccaacaagggtatcgacaagctcccctcccactgccagggcggcgtccgagctgcctgcaacgtctacgcctccatcggtaccaagctcaagtcctacaagcaccactacccctctcgagcccacgtcggtaactccaagcgagtcgagattgctctgctgtccgtctacaacctgtacaccgcccccattgccacctcctccaccacccactgccgacagggcaagatgcgaaacctcaacacgatctaaaggatataaaagtgcatgcacgagagtgtaataaatttataatcgaacgactgacaggccagtgcgatccacgagtctggccggtgacacgagcaccatgtagataacacgacgacaaatgacggtgacaagagcatgggcggagtttgactgatggaggatgtagatctggatttccgctgggatgtcagtatgaaatgctgaaattctaccgagtgtcctcgttatcgtgtggtatctgctatatctgcaaaggccatgctggtgtgttgtggttatcgtgtgtggagtgtagatgtttctgcaccttctttcgcttgcactgttcccagactccaagatttgtatacaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacacactagtggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggatgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagatgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacactatctatgtcgggtgcggagaaagaggtaatgaaatggtctagaatactcactttccgctcactataaatagacatacgcggctgagaacggacataattacgctacgtttctttaacccagactgagtgagtagtatcacagaggttggctgcgacgttcactttatcccttgaaaaatgtgctgaaaatgacgcaaaaagtatggagcaaactccggtcttgataccacgctgctcgcctgagatggaaggttgcagctgcattgcatgaatgcaggtttggtcagaggattctcaggggttttgataggcttgtcaggaacagaagactcgtacttgtacatgtagttggtacacacatttaagtgccatcgatccggtcatatatatctcctggttgccttcttctctgcactcgttcactccaaccggagtgttccatccaagtcttccagcacaccctgactctacagtaactttgaacgtcataaattcctatatacagtacacttagagagccttctcaagagcatcaaactgctggagaatggcggacttggcagttcctccaatggcggttcgtcgctcgacagactgctcaaagtcaaagaccttggcaacgtcctcctcgaatcgctcgtcaatcttcttgtactgctccagggtcagcttgtcgataccagacaccttgagctcttcggcagttcgaacagcctcaccagagatgtggtgggtctctcggaaggggacgccctttcggaccaggtagtcggccagatcggtggccagcatgtccatggtaagagcaccctccatcttctccttgttgatggagagggtggacatgacgccggtggcaatctggatggagtgagacacggtggtgaggttgtcaaacaggggctccttgtcctcctgcagatccttgttgtaggtggaaggagtggacttgacggacatcatgaagccggccagctggccaaaagctcggccagacttgcctcgaagaagctccagagagtccgggttcttcttctggggcatgagagaagaaccggtggagtaggcatcggcaagctggatgaagccaaactcggcagtggagtagatgatgagatcctcagcaaatcgagaaatgtggttcatgagcagagagccccagaagagagtctcggtgacaaagtctcggtcaccgacaccaacaagggagttaccgatgatgccgccaaagccaagctcggtggcaatgtactctcggtcaatgccaaaggggtggccagccagagctccacagcccagaggagacatgttgactcgctcaatgagctggtccagtcgctgcaggtcggaggtgaagtaagtagcgtaagatgaaagccagtgggcccatcggatgggctgggctcgctgaagatgggtgtatcccggcatgagcacgtccatctcagtcttacctcggttgataagcaccttgatgaagtcggccagcaggtttcggatctctcccagctcctttcgcacatacagtcgcatgtcggtagcgacttgatcgtttcgagatcggccagtgtgcaccttgccagagatgtctcggccaataacggccttggtggcctctagatggcctccttggcccggtctccatatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 4 - DNA, 13246 bp, synthetic construct
  source 1..13246: mol_type = other DNA
ggttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctggcctaggaagcgacttccaatcgctttgcatatccagtaccacacccacaggcgtttgtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaacaatggacaagaagtactccatcggtttggacattggtactaactctgtcggctgggccgtcatcaccgacgagtacaaggttccctccaagaagttcaaggtccttggcaacaccgaccgacactctatcaagaagaacctgatcggtgctctgctgttcgactctggcgagactgccgaggccacccgactgaagcgaaccgctcgacgccgatacacccgacgaaagaaccgaatctgttacctccaggagatcttcagcaacgagatggctaaggtcgacgactccttcttccaccgactcgaggagtctttcctggtcgaagaggataagaagcacgagcgacaccccatcttcggcaacattgttgatgaggttgcctaccatgagaagtaccccaccatctaccacctccgaaagaagctcgtcgactccactgacaaggctgacctccgactcatctaccttgctctcgcccacatgatcaagttccgaggtcacttcctcattgagggtgatctcaaccccgacaactccgacgttgacaagctgttcatccagctcgtccagacctacaaccagctctttgaggagaaccctatcaacgcttctggtgttgacgccaaggccattctctccgcccgactctctaagtcccgacgactcgagaacctcattgcccagctgcccggcgagaagaagaacggcctcttcggtaacctgattgctctctctcttggtctgacccccaacttcaagtccaactttgacctcgccgaggacgccaagctccagctgtccaaggacacctacgatgacgatctggacaacctcctggcccagatcggtgaccagtacgccgatctcttccttgccgccaagaacctctccgacgccatcctgctctccgacatcctccgagtcaacaccgagattaccaaggctcctctgtctgcctctatgatcaagcgatacgacgagcaccaccaggatctcactcttctcaaggctctcgtccgacagcagctccccgagaagtacaaggagattttctttgaccagtccaagaacggttacgctggctacattgacggtggtgcttcccaggaagagttttacaagttcatcaagcctattctggagaagatggacggtaccgaggagctgctcgtcaagctcaaccgagaggacctccttcgaaagcagcgaaccttcgataacggctccatcccccaccagatccacctgggtgagctccacgccattctccgaagacaagaggacttctaccccttcctaaaggataaccgagagaagatcgagaagattctcaccttccgaatcccctactacgtcggtcccctcgctcgaggtaactcccgatttgcttggatgacccgaaagtccgaggagactatcaccccctggaactttgaagaggtagtcgacaagggtgcctccgcccagtctttcattgagcggatgaccaacttcgataagaacctccccaacgagaaggtccttcccaagcactctctcctctacgagtacttcaccgtctacaacgagctgaccaaggtcaagtacgttaccgagggcatgcgaaagcccgctttcctctctggtgagcagaagaaggccattgtcgacctcctgttcaagactaaccgaaaagtcaccgtcaagcagctcaaggaagactacttcaagaagattgagtgcttcgactccgtcgagatttccggtgtcgaggaccgattcaacgcctccctcggcacctaccacgatcttctgaagatcatcaaggacaaggactttcttgataacgaggagaacgaggacattctcgaggacatcgtcctcaccctcacccttttcgaggatcgagagatgatcgaggagcgactcaagacctacgcccatctcttcgacgacaaggtcatgaagcaactcaagcgacgacgatacactggctggggccgactttcccgaaagctcatcaacggcatccgagacaagcagtctggcaagaccatcctggacttcctgaagtccgacggtttcgccaaccgaaacttcatgcagctcatccacgacgactctcttaccttcaaagaggatatccagaaggcccaggtttctggccagggcgactccctccacgagcacattgccaacctcgccggatcccccgccatcaaaaagggtatcctccagaccgtcaaggttgtcgacgaactcgtgaaggtcatgggccgacacaagcccgagaacatcgttatcgagatggcccgagagaaccagaccacccagaagggtcagaagaactcccgagagcgaatgaagcgaatcgaagagggtatcaaggagctcggttcccagattctcaaggagcaccccgtcgagaacacccagctccagaacgagaaactctacctgtactacctccagaatggccgagacatgtacgttgaccaggagctcgacatcaaccgactctccgactacgacgtcgaccacattgttcctcagtccttcctcaaggacgactccatcgacaacaaggttctgacccgatctgacaagaaccgaggtaagtccgacaacgttccctccgaagaggtcgttaagaagatgaagaactactggcgacagcttctcaacgccaaactgatcacccagcgaaagtttgacaacctcaccaaggccgagcgaggtggtctgtccgagctggacaaggccggcttcattaagcgacagctggtcgagactcgacagatcaccaagcacgtcgcccagatcctcgactcccgaatgaacaccaagtacgacgagaacgacaagctcatccgggaggtcaaggtcatcaccctgaagtctaagcttgtctccgacttccgaaaggacttccagttctacaaggtccgagagatcaacaactaccaccacgcccacgacgcctacctcaacgccgttgttggtaccgccctcatcaagaagtatcccaagctcgagtccgagttcgtttacggcgactacaaggtttacgatgtccgaaagatgattgccaagtccgagcaggagatcggtaaggccaccgccaagtactttttctactccaacatcatgaatttcttcaagaccgagatcactctcgccaacggtgagattcgaaagcgacccctgattgagactaatggtgagactggtgagatcgtctgggataagggccgagacttcgccaccgtccgaaaggtcctgtccatgccccaggtcaacattgtcaagaagaccgaggtccagaccggtggcttctccaaggagtccattctccccaagcgaaactccgacaaactcatcgcccgtaagaaggactgggatccgaagaagtacggtggtttcgattctcccaccgttgcctactccgtcctcgttgttgctaaagtcgagaagggtaagtctaagaaactcaagtccgtgaaggagctactcggtatcaccatcatggagcgatcttcttttgagaagaaccccattgacttcctcgaggccaagggttacaaagaggtcaagaaggacctgattatcaagctgcccaagtactccctctttgagctcgagaacggccgaaagcgaatgctggcttccgctggtgagctgcagaagggcaacgagctcgctctgccctccaagtacgtcaacttcctctacctggcctcccactacgagaagctcaagggctcccccgaggacaacgagcagaagcagctgttcgttgagcagcacaagcactacctcgacgagatcatcgagcagatctccgagttctccaagcgagtcatcctcgctgacgccaaccttgataaggttctctctgcttacaacaagcaccgggacaagcccatccgagagcaggccgagaatatcatccacctcttcactctcaccaacctcggcgctcctgctgccttcaagtacttcgacaccaccattgaccgaaagaggtacacctccaccaaggaagtcctcgacgccaccctgatccaccagtccatcaccggcctctacgaaacccgaatcgacctctcccagctcggcggtgactctcgagccgaccccaagaagaagcgaaaagtctaaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattattacctcttgcccatcgaacgtacaagtactcctctgttctctccttcctttgctttgtgcacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacaatgggtactctgatgagtccgtgaggacgaaacgagtaagctcgtcagtaccacacattgcagacagttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaacttcgagctaatccagtagcttacgttacccaggggcaggtcaactggctagccacgagtctgtcccaggtcgcaatttagtgtaataaacaatatatatattgagtctaaagggaattgtagctattgtgattgtgtgattttcgtcttgctggttcttattgtgtcccattcgtttcatcctgatgaggacccctggaaccggtgttttcttagtctctgcaatcgctagtcttgttgctatgacagttgcgtcgacactattcaggtcatctatcggttattctgatattataatacctccggatcgatgtacctgatttatacttgcagcaatgtttacttcttatcgttggaccccgtcttcaattacacttcccaactgggaacacccctctttatcgacccattttaggtaatttaccctagcccattgtctccataaggaatattaccctaacccacagtccagggtgcccaggtccttctttggccaaattttaacttcggtcctatggcacagcggtagcgcgtgagattgcaaatcttaaggtcccgagttcgaatctcggtgggacctagttgaaaaatacctctaatgcgccgatggtttagtggtaaaatccatcgttgccatcgatgggcccccggttcgattccgggtcggcgcaggttgacgtgggcacgtagccaatgacaggttttagagctagaaatagcaagttaaaataaggctagtccgttatcaacttgaaaaagtggcaccgagtcggtgcttttttttttcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgtctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatgggtacctgagaacatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccgaattttcagaacctaatttatctgttacccggcctgtggctcgcacagcttaaaaatggtcaaactttccccttcttgtctttttttcctcacattcatcaggttcttgtcttgatctttcaagtgagtattaattaccgaccttggttcttcattgggagagcattggaagccgtggtgcagcaaccacaaaacggttcttccccttcgataccttcttgcctgcctttcaatacaagtcggctcgattagcggtggtcgcccccgccagcggagaacatggaactaacccagaatgagagctaagtggagaaagaagagagtcagacgactcaagcgaaagcgccgcaaggtccgagctcgatccaaataagcggtttttaacggagatttaacactaaatcgaagaacttttcccgtttcatttgcgaatgagctcgttaacaaaatcccccagtttttttatccagctgtaaggattgacattagtaatgaattattgtttggtatatttaaatctgtagttcctttctgtccgtgtcggcaactgtcgtactcgtgatttacttgtattgacgaatacttactgtagcgcactctgctgctactggtcgtaaggatgtgctatttcggtgtatggtgggttttttgggggtcggaaccgaagactgttacacgggcacggctcgttgtgtacacgcacagagctcttgcgagtcatgttgtagctagctcgtcgtgttcaggaactgttcgatggttcggagagagtcgccgcccagaacatacgcgcaccgatgtcagcagacagccttattacaagtatattcaagcaagtatatccgtagggtgcgggtgatttggatctaaggttcgtactcaacactcacgagcagcttgcctatgttacatccttttatcagacataacataattggagtttacttacacacggggtgtacctgtatgagcaccacctacaattgtagcactggtacttgtacaaagaatttattcgtacgaatcacagggacggccgccctcaccgaaccagcgaatacctcagcggtcccctgcagtgactcaacaaagcgatatgaacatcttgcgatggtatcctgctgatagtttttactgtacaaacacctgtgtagctccttctagcatttttaagttattcacacctcaaggggagggataaattaaataaattccaaaagcgaagatcgagaaactaaattaaaattccaaaaacgaagttggaacacaaccccccgaaaaaaaacaacaaacaaaaaacccaacaaaataaacaaaaacaaaataaatatataactaccagtatctgactaaaagttcaaatactcgtacttacaacaaatagaaatgagccggccaaaattctgcagaaaaaaatttcaaacaagtactggtataattaaattaaaaaacacatcaaagtatcataacgttagttattttattttatttaataaaagaaaacaacaagatgggctcaaaactttcaacttatacgatacataccaaataacaatttagtatttatctaagtgcttttcgtagataatggaatacaaatggatatccagagtatacacatggatagtatacactgacacgacaattctgtatctctttatgttaactactgtgaggcattaaatagagcttgatatataaaatgttacatttcacagtctgaacttttgcagattacctaatttggtaagatattaattatgaactgaaagttgatggcatccctaaatttgatgaaagatgaaattgtaaatgaggtggtaaaagagctacagtcgttttgttttgagataccatcatctctaacgaaatatctattaaaaatctcagtgtgatcatgagtcattgccatcctggaaaatgtcatcatggctgatatttctaactgtttacttgagataaatatatatttacaagaacttcccttgaaattaatttagatataaaatgtttgcgggcaagttactacgaggaataaattatatctaga

SEQ ID NO: 5 - DNA, 13246 bp, synthetic construct
  source 1..13246: mol_type = other DNA
ggttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctggcctaggaagcgacttccaatcgctttgcatatccagtaccacacccacaggcgtttgtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaacaatggacaagaagtactccatcggtttggacattggtactaactctgtcggctgggccgtcatcaccgacgagtacaaggttccctccaagaagttcaaggtccttggcaacaccgaccgacactctatcaagaagaacctgatcggtgctctgctgttcgactctggcgagactgccgaggccacccgactgaagcgaaccgctcgacgccgatacacccgacgaaagaaccgaatctgttacctccaggagatcttcagcaacgagatggctaaggtcgacgactccttcttccaccgactcgaggagtctttcctggtcgaagaggataagaagcacgagcgacaccccatcttcggcaacattgttgatgaggttgcctaccatgagaagtaccccaccatctaccacctccgaaagaagctcgtcgactccactgacaaggctgacctccgactcatctaccttgctctcgcccacatgatcaagttccgaggtcacttcctcattgagggtgatctcaaccccgacaactccgacgttgacaagctgttcatccagctcgtccagacctacaaccagctctttgaggagaaccctatcaacgcttctggtgttgacgccaaggccattctctccgcccgactctctaagtcccgacgactcgagaacctcattgcccagctgcccggcgagaagaagaacggcctcttcggtaacctgattgctctctctcttggtctgacccccaacttcaagtccaactttgacctcgccgaggacgccaagctccagctgtccaaggacacctacgatgacgatctggacaacctcctggcccagatcggtgaccagtacgccgatctcttccttgccgccaagaacctctccgacgccatcctgctctccgacatcctccgagtcaacaccgagattaccaaggctcctctgtctgcctctatgatcaagcgatacgacgagcaccaccaggatctcactcttctcaaggctctcgtccgacagcagctccccgagaagtacaaggagattttctttgaccagtccaagaacggttacgctggctacattgacggtggtgcttcccaggaagagttttacaagttcatcaagcctattctggagaagatggacggtaccgaggagctgctcgtcaagctcaaccgagaggacctccttcgaaagcagcgaaccttcgataacggctccatcccccaccagatccacctgggtgagctccacgccattctccgaagacaagaggacttctaccccttcctaaaggataaccgagagaagatcgagaagattctcaccttccgaatcccctactacgtcggtcccctcgctcgaggtaactcccgatttgcttggatgacccgaaagtccgaggagactatcaccccctggaactttgaagaggtagtcgacaagggtgcctccgcccagtctttcattgagcggatgaccaacttcgataagaacctccccaacgagaaggtccttcccaagcactctctcctctacgagtacttcaccgtctacaacgagctgaccaaggtcaagtacgttaccgagggcatgcgaaagcccgctttcctctctggtgagcagaagaaggccattgtcgacctcctgttcaagactaaccgaaaagtcaccgtcaagcagctcaaggaagactacttcaagaagattgagtgcttcgactccgtcgagatttccggtgtcgaggaccgattcaacgcctccctcggcacctaccacgatcttctgaagatcatcaaggacaaggactttcttgataacgaggagaacgaggacattctcgaggacatcgtcctcaccctcacccttttcgaggatcgagagatgatcgaggagcgactcaagacctacgcccatctcttcgacgacaaggtcatgaagcaactcaagcgacgacgatacactggctggggccgactttcccgaaagctcatcaacggcatccgagacaagcagtctggcaagaccatcctggacttcctgaagtccgacggtttcgccaaccgaaacttcatgcagctcatccacgacgactctcttaccttcaaagaggatatccagaaggcccaggtttctggccagggcgactccctccacgagcacattgccaacctcgccggatcccccgccatcaaaaagggtatcctccagaccgtcaaggttgtcgacgaactcgtgaaggtcatgggccgacacaagcccgagaacatcgttatcgagatggcccgagagaaccagaccacccagaagggtcagaagaactcccgagagcgaatgaagcgaatcgaagagggtatcaaggagctcggttcccagattctcaaggagcaccccgtcgagaacacccagctccagaacgagaaactctacctgtactacctccagaatggccgagacatgtacgttgaccaggagctcgacatcaaccgactctccgactacgacgtcgaccacattgttcctcagtccttcctcaaggacgactccatcgacaacaaggttctgacccgatctgacaagaaccgaggtaagtccgacaacgttccctccgaagaggtcgttaagaagatgaagaactactggcgacagcttctcaacgccaaactgatcacccagcgaaagtttgacaacctcaccaaggccgagcgaggtggtctgtccgagctggacaaggccggcttcattaagcgacagctggtcgagactcgacagatcaccaagcacgtcgcccagatcctcgactcccgaatgaacaccaagtacgacgagaacgacaagctcatccgggaggtcaaggtcatcaccctgaagtctaagcttgtctccgacttccgaaaggacttccagttctacaaggtccgagagatcaacaactaccaccacgcccacgacgcctacctcaacgccgttgttggtaccgccctcatcaagaagtatcccaagctcgagtccgagttcgtttacggcgactacaaggtttacgatgtccgaaagatgattgccaagtccgagcaggagatcggtaaggccaccgccaagtactttttctactccaacatcatgaatttcttcaagaccgagatcactctcgccaacggtgagattcgaaagcgacccctgattgagactaatggtgagactggtgagatcgtctgggataagggccgagacttcgccaccgtccgaaaggtcctgtccatgccccaggtcaacattgtcaagaagaccgaggtccagaccggtggcttctccaaggagtccattctccccaagcgaaactccgacaaactcatcgcccgtaagaaggactgggatccgaagaagtacggtggtttcgattctcccaccgttgcctactccgtcctcgttgttgctaaagtcgagaagggtaagtctaagaaactcaagtccgtgaaggagctactcggtatcaccatcatggagcgatcttcttttgagaagaaccccattgacttcctcgaggccaagggttacaaagaggtcaagaaggacctgattatcaagctgcccaagtactccctctttgagctcgagaacggccgaaagcgaatgctggcttccgctggtgagctgcagaagggcaacgagctcgctctgccctccaagtacgtcaacttcctctacctggcctcccactacgagaagctcaagggctcccccgaggacaacgagcagaagcagctgttcgttgagcagcacaagcactacctcgacgagatcatcgagcagatctccgagttctccaagcgagtcatcctcgctgacgccaaccttgataaggttctctctgcttacaacaagcaccgggacaagcccatccgagagcaggccgagaatatcatccacctcttcactctcaccaacctcggcgctcctgctgccttcaagtacttcgacaccaccattgaccgaaagaggtacacctccaccaaggaagtcctcgacgccaccctgatccaccagtccatcaccggcctctacgaaacccgaatcgacctctcccagctcggcggtgactctcgagccgaccccaagaagaagcgaaaagtctaaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattattacctcttgcccatcgaacgtacaagtactcctctgttctctccttcctttgctttgtgcacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacaatgacgagtctgatgagtccgtgaggacgaaacgagtaagctcgtcactcgtctacgcatgcctgggttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaacttcgagctaatccagtagcttacgttacccaggggcaggtcaactggctagccacgagtctgtcccaggtcgcaatttagtgtaataaacaatatatatattgagtctaaagggaattgtagctattgtgattgtgtgattttcgtcttgctggttcttattgtgtcccattcgtttcatcctgatgaggacccctggaaccggtgttttcttagtctctgcaatcgctagtcttgttgctatgacagttgcgtcgacactattcaggtcatctatcggttattctgatattataatacctccggatcgatgtacctgatttatacttgcagcaatgtttacttcttatcgttggaccccgtcttcaattacacttcccaactgggaacacccctctttatcgacccattttaggtaatttaccctagcccattgtctccataaggaatattaccctaacccacagtccagggtgcccaggtccttctttggccaaattttaacttcggtcctatggcacagcggtagcgcgtgagattgcaaatcttaaggtcccgagttcgaatctcggtgggacctagttgaaaaatacctctaatgcgccgatggtttagtggtaaaatccatcgttgccatcgatgggcccccggttcgattccgggtcggcgcaggttgacgtcgaacgtcttgtcttccgaagttttagagctagaaatagcaagttaaaataaggctagtccgttatcaacttgaaaaagtggcaccgagtcggtgcttttttttttcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgtctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatgggtacctgagaacatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccgaattttcagaacctaatttatctgttacccggcctgtggctcgcacagcttaaaaatggtcaaactttccccttcttgtctttttttcctcacattcatcaggttcttgtcttgatctttcaagtgagtattaattaccgaccttggttcttcattgggagagcattggaagccgtggtgcagcaaccacaaaacggttcttccccttcgataccttcttgcctgcctttcaatacaagtcggctcgattagcggtggtcgcccccgccagcggagaacatggaactaacccagaatgagagctaagtggagaaagaagagagtcagacgactcaagcgaaagcgccgcaaggtccgagctcgatccaaataagcggtttttaacggagatttaacactaaatcgaagaacttttcccgtttcatttgcgaatgagctcgttaacaaaatcccccagtttttttatccagctgtaaggattgacattagtaatgaattattgtttggtatatttaaatctgtagttcctttctgtccgtgtcggcaactgtcgtactcgtgatttacttgtattgacgaatacttactgtagcgcactctgctgctactggtcgtaaggatgtgctatttcggtgtatggtgggttttttgggggtcggaaccgaagactgttacacgggcacggctcgttgtgtacacgcacagagctcttgcgagtcatgttgtagctagctcgtcgtgttcaggaactgttcgatggttcggagagagtcgccgcccagaacatacgcgcaccgatgtcagcagacagccttattacaagtatattcaagcaagtatatccgtagggtgcgggtgatttggatctaaggttcgtactcaacactcacgagcagcttgcctatgttacatccttttatcagacataacataattggagtttacttacacacggggtgtacctgtatgagcaccacctacaattgtagcactggtacttgtacaaagaatttattcgtacgaatcacagggacggccgccctcaccgaaccagcgaatacctcagcggtcccctgcagtgactcaacaaagcgatatgaacatcttgcgatggtatcctgctgatagtttttactgtacaaacacctgtgtagctccttctagcatttttaagttattcacacctcaaggggagggataaattaaataaattccaaaagcgaagatcgagaaactaaattaaaattccaaaaacgaagttggaacacaaccccccgaaaaaaaacaacaaacaaaaaacccaacaaaataaacaaaaacaaaataaatatataactaccagtatctgactaaaagttcaaatactcgtacttacaacaaatagaaatgagccggccaaaattctgcagaaaaaaatttcaaacaagtactggtataattaaattaaaaaacacatcaaagtatcataacgttagttattttattttatttaataaaagaaaacaacaagatgggctcaaaactttcaacttatacgatacataccaaataacaatttagtatttatctaagtgcttttcgtagataatggaatacaaatggatatccagagtatacacatggatagtatacactgacacgacaattctgtatctctttatgttaactactgtgaggcattaaatagagcttgatatataaaatgttacatttcacagtctgaacttttgcagattacctaatttggtaagatattaattatgaactgaaagttgatggcatccctaaatttgatgaaagatgaaattgtaaatgaggtggtaaaagagctacagtcgttttgttttgagataccatcatctctaacgaaatatctattaaaaatctcagtgtgatcatgagtcattgccatcctggaaaatgtcatcatggctgatatttctaactgtttacttgagataaatatatatttacaagaacttcccttgaaattaatttagatataaaatgtttgcgggcaagttactacgaggaataaattatatctaga

SEQ ID NO: 6 - DNA, 9918 bp, synthetic construct
  source 1..9918: mol_type = other DNA
gcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccgaattttcagaacctaatttatctgttacccggcctgtggctcgcacagcttaaaaatggtcaaactttccccttcttgtctttttttcctcacattcatcaggttcttgtcttgatctttcaagtgagtattaattaccgaccttggttcttcattgggagagcattggaagccgtggtgcagcaaccacaaaacggttcttccccttcgataccttcttgcctgcctttcaatacaagtcggctcgattagcggtggtcgcccccgccagcggagaacatggaactaacccagaatgagagctaagtggagaaagaagagagtcagacgactcaagcgaaagcgccgcaaggtccgagctcgatccaaataagcggtttttaacggagatttaacactaaatcgaagaacttttcccgtttcatttgcgaatgagctcgttaacaaaatcccccagtttttttatccagctgtaaggattgacattagtaatgaattattgtttggtatatttaaatctgtagttcctttctgtccgtgtcggcaactgtcgtactcgtgatttacttgtattgacgaatacttactgtagcgcactctgctgctactggtcgtaaggatgtgctatttcggtgtatggtgggttttttgggggtcggaaccgaagactgttacacgggcacggctcgttgtgtacacgcacagagctcttgcgagtcatgttgtagctagctcgtcgtgttcaggaactgttcgatggttcggagagagtcgccgcccagaacatacgcgcaccgatgtcagcagacagccttattacaagtatattcaagcaagtatatccgtagggtgcgggtgatttggatctaaggttcgtactcaacactcacgagcagcttgcctatgttacatccttttatcagacataacataattggagtttacttacacacggggtgtacctgtatgagcaccacctacaattgtagcactggtacttgtacaaagaatttattcgtacgaatcacagggacggccgccctcaccgaaccagcgaatacctcagcggtcccctgcagtgactcaacaaagcgatatgaacatcttgcgatggtatcctgctgatagtttttactgtacaaacacctgtgtagctccttctagcatttttaagttattcacacctcaaggggagggataaattaaataaattccaaaagcgaagatcgagaaactaaattaaaattccaaaaacgaagttggaacacaaccccccgaaaaaaaacaacaaacaaaaaacccaacaaaataaacaaaaacaaaataaatatataactaccagtatctgactaaaagttcaaatactcgtacttacaacaaatagaaatgagccggccaaaattctgcagaaaaaaatttcaaacaagtactggtataattaaattaaaaaacacatcaaagtatcataacgttagttattttattttatttaataaaagaaaacaacaagatgggctcaaaactttcaacttatacgatacataccaaataacaatttagtatttatctaagtgcttttcgtagataatggaatacaaatggatatccagagtatacacatggatagtatacactgacacgacaattctgtatctctttatgttaactactgtgaggcattaaatagagcttgatatataaaatgttacatttcacagtctgaacttttgcagattacctaatttggtaagatattaattatgaactgaaagttgatggcatccctaaatttgatgaaagatgaaattgtaaatgaggtggtaaaagagctacagtcgttttgttttgagataccatcatctctaacgaaatatctattaaaaatctcagtgtgatcatgagtcattgccatcctggaaaatgtcatcatggctgatatttctaactgtttacttgagataaatatatatttacaagaacttcccttgaaattaatttagatataaaatgtttgcgggcaagttactacgaggaataaattatatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccagagacgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcgctagacacaaaaatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcgtgcaattaacagatagtttgccggtgataattctcttaacctcccacactcctttgacataacgatttatgtaacgaaactgaaatttgaccagatattgttgtaaatagaaaatctggcttgtaggtggcaaatctagtggaaggactatacgggatctcaaaagctgcaaagaatgcacgcggatagtaacaaacattcgcagcacagaaagtacgaaaagctgtttgatttgctgcagtgaaccaaacagcacagacgaatgagggaatgtatatatgtatatagaaattgcatggttgttatatcgacggtaataataaactaacatgcaacattccattacgcaaccttatattggacacgtgccataatcaccacccataggccgagagtcagtaggagtactccagacacagttcaccgctctcggctcggtcggtggcaaacactacagcgaatgacccacatctaccacacacgctcagtataataaatccattccaatatcatatataatataaataacgcgtctaatcgccatcttccagcaggcgcaccattgcccctgtttcactatccagggtacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagttacccccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacgaacctggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcctgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatttttgtgtctagatgtttagtatttgttgtgtgtgtgggaaggaagacctttgggaggggccggctggtcttatatatttttttgcctctcccttgggtctgttttgtgttgtggggggggctcccaattaggcgtttcctatcaaccggtccctgatatacatgccgtgctgtgatatgctgcactacgtggtgctgggaccggaaataccacacaaaatgcgaggcttttacatcgctcccaaattttgacacttcaccatatgccaattttgagacttggttacctcccaaccactcccaactcgccctaaccctaaagcttattagccgggaactcagaaactggggtcgtgccctgggtaagcgaaaataggtgattgggggcgattacggaagatatggggggatttaaaaggttgggttacggaaaaggcactactgcgacattactggtcgcatttcttcttctgatcgacgccgggaaatgtacctccataaagaatcagcctcagaaacccaccacaccagttcgaatcccgcctgtgtaacattttccgaaggaataaaagagtataattggtgaatactgaagattaaaagatgaaaatagccttcaaattgatgccaccgttttataaaccacgatcagcacacctgttgctgcctcaatcatttgttcttgttcagtttacacatggagcaagtttgtcctaatcggagcaaccctaaacctagttagggggcagcgcgtcattccattaaccccaaccctattatccaaagcagaacacctaacccgaatacaaaaaaaagagaggaaacctaaccctagcattaactaccctaaccgggacacaccctaacccccaaataagctcagttgttgccgttgccgtcacctgggcaaccagccccaacccacagaatcgttggtatccacgtatattctaccccagataagttt

SEQ ID NO: 7 - DNA, 9568 bp, synthetic construct
  source 1..9568: mol_type = other DNA
gttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccagatagtgcaatcacatgttgctactgtacctgctgtggaccacgcacggcggaacgtaccgtacaaatattttcttgctcacatgactctctctcggccgcgcacgccggtggcaaattgctcttgcattggctctgtctctagacgtccaaaccgtccaaagtggcagggtgacgtgatgcgacgcacgaaggagatggcccggtggcgaggaaccggacacggcgagccggcgggaaaaaaggcggaaaacgaaaagcgaagggcacaatctgacggtgcggctgccaccaacccaaggaggctattttgggtcgctttccatttcacattcgccctcaatggccactttgcggtggtgaacatggtttctgaaacaaccccccagaattagagtatattgatgtgtttaagattgggttgctatttggccattgtgggggagggtagcgacgtggaggacattccagggcgaattgagcctagaaagtggtaccattccaaccgtctcagtcgtccgaattgatcgctataactatcacctctctcacatgtctacttccccaaccaacatccccaacctcccccacactaaagttcacgccaataatgtaggcactctttctgggtgtgggacagcagagcaatacggaggggagattacacaacgagccacaattggggagatggtagccatctcactcgacccgtcgacttttggcaacgctcaattacccaccaaatttgggctggagttgaggggaccgtgttccagcgctgtaggaccagcaacacacacggtatcaacagcaaccaacgcccccgctaatgcacccagtactgcgcaggtgtgggccaggtgcgttccagatgcgagttggcgaaccctaagccgacagtgtactttttgggacgggcagtagcaatcgtgggcggagaccccggtgtatataaaggggtggagaggacggattattagcaccaacacacacacttatactacatgctagacacaaaaatggaccctaccggagacgttactgctagccctcgacctcaaaccaccattcctgtccgacaagcactctggggacttagccttgctggagccatcatcgccgcatgggtttttatgcacattggtttcgttttttttgccccccttgatcctatcgttctcgccctcgccccagttattattcttcttcaatcctggctttctgttggtctttttattatttctcacgacgcaattcacggttccctcgcccctggacgacccgcctttaatagagccatgggacgactctgcatgacactttacgccggtttcgactttgaccgtatggccgctgcacatcaccgacatcacagatcccctggaaccgccgctgaccccgatttttctgttgactcccctgatcgacctctcccttggtttggagctttcttccgacgttactttggctggagaccttttcttaccgttaacgctgtcgtctttacctactggcttgttcttggagctaaccctgttaatattgttctcttttatggcgttcctgcactcctttccgccggacagctcttttactttggtacatttctccctcaccgacacgaacgacaaggctttgctgatcaccaccgagcacgatccgtccgatccccttacatgctttctcttgttacttgttaccactttggaggctatcatcacgaacatcatctctttccacacgaaccctggtggcgcctgcctcaacgaggaggttgggaacgtgacagacgaaagagaaccggcccttaacgcgtgttttttgatcaatgatccaatggctttcacatacccccccacgcctataattaaaacacagagaaatataatctaacttaataaatattacggagaatctttcgagtgttcagcagaaatatagccattgtaacaaaagccggctatcgaccgctttatcgaagaatatttcccgccccccagtggccaaacgatatttaccgttcgtataatgtatgctatacgaagttataccggtctcgtagtgttcacgttcagttcacggtgagcttaaaactatcttcaagaagagatttgagacctgatttatacttgcagcaatgtttacttcttatcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgtctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatgggtacctgagaacatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccataacttcgtataatgtatgctatacgaacggtacccgggcaattctgcagatatcggaggctcttcctccacctccgaggtcggagactttgttgccaacaaggtcaaggagctgctcaagaaggagtaagtcgtttctacgacgcattgatggaaggagcaaactgacgcgcctgcgggttggtctaccggcagggtccgctagtgtataagactctataaaaagggcccagccctgctaatgaaatgatgatttataatttaccggtgtagcaaccttgactagaagaagcagattgggtgtgtttgtagtggaggacagtggagccgaattccagcacactggcggccgttactagtttgccacctacaagccagattttctatttacaacaatatctggtcaaatttcagtttcgttacataaatcgttatgtcaaaggagtgtgggaggttaagagaattatcaccggcaaactatctgttaattgcacgcgttaagggccggttctctttcgtctgtcacgttcccaacctcctcgttgaggcaggcgccaccagggttcgtgtggaaagagatgatgttcgtgatgatagcctccaaagtggtaacaagtaacaagagaaagcatgtaaggggatcggacggatcgtgctcggtggtgatcagcaaagccttgtcgttcgtgtcggtgagggagaaatgtaccaaagtaaaagagctgtccggcggaaaggagtgcaggaacgccataaaagagaacaatattaacagggttagctccaagaacaagccagtaggtaaagacgacagcgttaacggtaagaaaaggtctccagccaaagtaacgtcggaagaaagctccaaaccaagggagaggtcgatcaggggagtcaacagaaaaatcggggtcagcggcggttccaggggatctgtgatgtcggtgatgtgcagcggccatacggtcaaagtcgaaaccggcgtaaagtgtcatgcagagtcgtcccatggctctattaaaggcgggtcgtccaggggcgagggaaccgtgaattgcgtcgtgagaaataataaaaagaccaacagaaagccaggattgaagaagaataataactggggcgagggcgagaacgataggatcaaggggggcaaaaaaaacgaaaccaatgtgcataaaaacccatgcggcgatgatggctccagcaaggctaagtccccagagtgcttgtcggacaggaatggtggtttgaggtcgagggctagcagtaacgtctccggtagggtccatttttgtgtctagcgaatgattcttatactcagaaggaaatgcttaacgatttcgggtgtgagttgacaaggagagagagaaaagaagaggaaaggtaattcggggacggtggtcttttatacccttggctaaagtcccaaccacaaagcaaaaaaattttcagtagtctattttgcgtccggcatgggttacccggatggccagacaaagaaactagtacaaagtctgaacaagcgtagattccagactgcagtaccctacgcccttaacggcaagtgtgggaaccgggggaggtttgatatgtggggtgaagggggctctcgccggggttgggcccgctactgggtcaatttggggtcaattggggcaattggggctgttttttgggacacaaatacgccgccaacccgtctctggtaccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacat

SEQ ID NO: 8 - DNA, 7740 bp, synthetic construct
  source 1..7740: mol_type = other DNA
gatccataacttcgtataatgtatgctatacgaagttatcccgggcaattctgcagatattcaattccagcacactggcggccgttactagtttgccacctacaagccagattttctatttacaacaatatctggtcaaatttcagtttcgttacataaatcgttatgtcaaaggagtgtgggaggttaagagaattatcaccggcaaactatctgttaattgcacgcgtttactctgccgatttcggcggcaggtcttgtgctcggctctcgctctgtgcgacaggtttaccgtcccgccgggctctggcggcgtctgctttaggtttccgaccgtgacgtctccgcagctctgcctgaagtgcttccggcttagctgcgtagagaaaaccgaaggagacgcaaccctctttgccattcactgcgtggtgcagccggtgtgcctggtaaagccgcttaaggtagcctctccgcggaatgtagttaaacggccatctccggtgcaccagaccgtcgtgcacgaggaaatagaacagaccgtagagcgtcatgcctgcgccaagccattgcagcggccaccagcctgctgttcccagtgcgatcagcacgatggcgagcagtgcgaacaccactgcgtagagatcgttgacctcaaaccagccgtgccgcggctcgtggtgcgaacggtgccagccccatccccagccgtgcatgatgtatttatgggcaagcgttgccacaatctccatcaggcaaaatgtcgtgatgacgataagcgtattccacagcacaagcatttttgtggctagcgaatgattcttatactcagaaggaaatgcttaacgatttcgggtgtgagttgacaaggagagagagaaaagaagaggaaaggtaattcggggacggtggtcttttatacccttggctaaagtcccaaccacaaagcaaaaaaattttcagtagtctattttgcgtccggcatgggttacccggatggccagacaaagaaactagtacaaagtctgaacaagcgtagattccagactgcagtaccctacgcccttaacggcaagtgtgggaaccgggggaggtttgatatgtggggtgaagggggctctcgccggggttgggcccgctactgggtcaatttggggtcaattggggcaattggggctgttttttgggacacaaatacgccgccaacccgtctctggtaccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccagagacgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcgctagccacaaaaatgcttgtgctgtggaatacgcttatcgtcatcacgacattttgcctgatggagattgtggcaacgcttgcccataaatacatcatgcacggctggggatggggctggcaccgttcgcaccacgagccgcggcacggctggtttgaggtcaacgatctctacgcagtggtgttcgcactgctcgccatcgtgctgatcgcactgggaacagcaggctggtggccgctgcaatggcttggcgcaggcatgacgctctacggtctgttctatttcctcgtgcacgacggtctggtgcaccggagatggccgtttaactacattccgcggagaggctaccttaagcggctttaccaggcacaccggctgcaccacgcagtgaatggcaaagagggttgcgtctccttcggttttctctacgcagctaagccggaagcacttcaggcagagctgcggagacgtcacggtcggaaacctaaagcagacgccgccagagcccggcgggacggtaaacctgtcgcacagagcgagagccgagcacaagacctgccgccgaaatcggcagagtaaacgcgtgcaattaacagatagtttgccggtgataattctcttaacctcccacactcctttgacataacgatttatgtaacgaaactgaaatttgaccagatattgttgtaaatagaaaatctggcttgtaggtggcaaactagtaacggccgccagtgtgctggaattgaatattataacttcgtataatgtatgctatacgaagttataccggtctcgtagtgttcacgttcagttcacggtgagcttaaaactatcttcaagaagagatttgagacctgatttatacttgcagcaatgtttacttcttatcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgttaggggcagggcatgctcatgtagagcgcctgctcgccgtccgaggcggtgccgtcgtacagggcggtgtccaggccgcagagggtgaaccccatccgccggtacgcgtggatcgccggtgcgttgacgttggtgacctccagccagaggtgcccggcgccccgctcgcgggcgaactccgtcgcgagccccatcaacgcgcgcccgaccccgtgcccccggtgctccggggcgacctcgatgtcctcgacggtcagccggcggttccagccggagtacgagacgaccacgaagcccgccaggtcgccgtcgtccccgtacgcgacgaacgtccgggagtccgggtcgccgtcctccccgtcgtccgattcgtcgtccgattcgtcgtcggggaacaccttggtcaggggcgggtccaccggcacctcccgcagggtgaagccgtccccggtggcggtgacgcggaagacggtgtcggtggtgaaggacccatccagtgcctcgatggcctcggcgtcccccgggacactggtgcggtaccggtaagccgtgtcgtcaagagtggtacccatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagg

SEQ ID NO: 9 - DNA, 8821 bp, synthetic construct
  source 1..8821: mol_type = other DNA
gttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccagagacgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcgctagccacaaaaatgctgtcgaagctgcagtcaatcagcgtcaaggcccgccgcgttgaactcgcccgcgacatcacgcggcccaaagtctgcctgcatgctcagcggtgctcgctcgttcggctgcgagtggcagcaccacagacagaggaggcggtgggaaccgtgcaggctgccggcgcgggcgatgagcacagcgccgatgttgcactccagcagcttgaccgggctatcgcagagcgtcgtgcccggcgcaaacgggagcagctctcataccaggctgccgccattgcagcatcaattggcgtgtcaggcattgccatcttcgccacctacctgagatttgccatgcacatgaccgtgggcggagcagtgccatggggtgaagtggctggcactctcctcttggtggttggtggcgcgctcggcatggagatgtatgcccgctatgcccacaaagccatctggcatgagtcgcctctgggctggctgctgcacaagagccaccacacacctcgcactggaccctttgaagccaacgacttgtttgcaatcatcaatggactgcccgccatgctcctgtgtacctttggcttctggctgcccaacgtcttgggtgcggcctgctttggcgcgggtctgggcatcacgctctacggcatggcatatatgtttgttcacgatggcctggtgcacagacgctttcccaccggacccatcgctggcctgccctacatgaagcgcctgacagtggcccaccagcttcaccacagcggcaagtacggtggcgcgccctggggcatgttcttgggtccacaggagctgcagcacattccaggtgctgcggaggaggtggagcgactcgtcctggaactggactggtcaaagcggtaaacgcgtgcaattaacagatagtttgccggtgataattctcttaacctcccacactcctttgacataacgatttatgtaacgaaactgaaatttgaccagatattgttgtaaatagaaaatctggcttgtaggtggcaaactagtaacggccgccagtgtgctggaattgaatatttaccgttcgtataatgtatgctatacgaagttataccggtctcgtagtgttcacgttcagttcacggtgagcttaaaactatcttcaagaagagatttgagacctgatttatacttgcagcaatgtttacttcttatcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgtctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatgggtacctgagaacatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccataacttcgtataatgtatgctatacgaacggtacccgggcaattctgcagatattcaattccagcacactggcggccgttactagtttgccacctacaagccagattttctatttacaacaatatctggtcaaatttcagtttcgttacataaatcgttatgtcaaaggagtgtgggaggttaagagaattatcaccggcaaactatctgttaattgcacgcgtttacactctgcgctttgaccagtccaggtctgccaccaagcggtccagctcctctgtggcacctgaaattgcctccagctcctgaggacccaggaacatgccccagggcgcgccatcatacttgccgctgtggtggatctggtgggccactgtcaggcgcttcatgtagggcaggtctgagatgggtccggtgggaaagcgccggtggacgaggccgtcatggatgaacatgtacgccataccgtacagggtgatgcccagaccagcaccaaagcatgaggcgccgaccatgccaggctcccagaaaccataatagcacagcagcatggctggcaggccattgatgatggcgaagaggtcattggcttcaaagggtccggtgcggggagtgtggtggctcttgtgcagcagccaacccaggggggactcatgccagagggccttgtgtgcataacgcgcatacatctccatgccaaacgcgccacctgccaccagtgagagggtgcccaagaggtcgctccaaggcatctcgccatctgatgtgatgtgcattgaaaaccgcgtgtaggtggcaaggatggcaaggcttgaaatgcccacggacgctgcaatggctgcagcctggtaggtgagctgctctcttttgcgttgagcgcggcgcagagcgatggatctgtcgagctgttggagggcgtcgtctgcagtttgctgattgcctgcaccgacagacggctgcacccgtgaggactccaccgcctctgtgtctggggctgcaacgcggagcacctgggccaatggctgtctttgagagggcatctgaggtcttgagatgtcacgaggcaatgcgatgcgccgtccttgcacacccattggctgcatcttcgacaaagccatttttgtggctagcgaatgattcttatactcagaaggaaatgcttaacgatttcgggtgtgagttgacaaggagagagagaaaagaagaggaaaggtaattcggggacggtggtcttttatacccttggctaaagtcccaaccacaaagcaaaaaaattttcagtagtctattttgcgtccggcatgggttacccggatggccagacaaagaaactagtacaaagtctgaacaagcgtagattccagactgcagtaccctacgcccttaacggcaagtgtgggaaccgggggaggtttgatatgtggggtgaagggggctctcgccggggttgggcccgctactgggtcaatttggggtcaattggggcaattggggctgttttttgggacacaaatacgccgccaacccgtctctggtaccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacat

SEQ ID NO: 10 - DNA, 6916 bp, synthetic construct
  source 1..6916: mol_type = other DNA
gttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccagagacgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcgctagccacaaaaatggagcgaactgagcgaaagcgaaaggagcgatacacttaccttgttgctgctattatgtcttctcttggtatcacttctcttgttgttgtgtccgtttactaccgattttcttgtcagtttgagggtggacagattcctgtttctgagatgcttggtacctttgctctttctatgggtaccgctgtgggtatggagttttgggctcgatggactcacaaggagctttggcacgcttctctttggcacatgcacgagtctcaccaccgacctggcgacggtcctttcgagcttaacgatgtcatccccatcattaacgccattcctgccattgctcttctctctctcggttttctcaacaagggttttttccctgacctttgttttggtgctggtcttggtattactgtttttggtattgcttacattctcgttcacgacggcctcgttcaccgacgatttcccgttggacccatcgctcacgtcccttaccttcagaaggttgctgccgctcacctccttcaccactctgatcgatttaacggagttccctttggtcttctccttggtcctaaggagcttgaggagattggtgctggtgagaaggaggattaaacgcgtgcaattaacagatagtttgccggtgataattctcttaacctcccacactcctttgacataacgatttatgtaacgaaactgaaatttgaccagatattgttgtaaatagaaaatctggcttgtaggtggcaaactagtaacggccgccagtgtgctggaattgaatatttaccgttcgtataatgtatgctatacgaagttataccggtctcgtagtgttcacgttcagttcacggtgagcttaaaactatcttcaagaagagatttgagacctgatttatacttgcagcaatgtttacttcttatcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgtctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatgggtacctgagaacatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccataacttcgtataatgtatgctatacgaacggtacccgggcaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacat

SEQ ID NO: 11 - DNA, 7942 bp, synthetic construct
  source 1..7942: mol_type = other DNA
cgcgtgcaattaacagatagtttgccggtgataattctcttaacctcccacactcctttgacataacgatttatgtaacgaaactgaaatttgaccagatattgttgtaaatagaaaatctggcttgtaggtggcaaactagtaacggccgccagtgtgctggaattgaatatttaccgttcgtataatgtatgctatacgaagttataccggtctcgtagtgttcacgttcagttcacggtgagcttaaaactatcttcaagaagagatttgagacctgatttatacttgcagcaatgtttacttcttatcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgtctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatgggtacctgagaacatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccataacttcgtataatgtatgctatacgaacggtacccgggcaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccagagacgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcgctagccacaaaaatgggtcgaaaggagtctgatgacggcgtggagcgaattgagggaggtgttgttgtcgtgaaccctaagcccaagaagggaattactgctaaggccattgacctccttgagaaggtcatcatcaagctgatgcacgattcttccaagccccttcactacctttctggcaactttgctcctaccgatgagactccccctctcaacgacctccctatcaagggccaccttcccgagtgccttaacggtgagtttgttcgagttggtcctaaccctaagtttgctcccgttgctggataccactggtttgacggcgatggcatgattcacggccttcgaattaaggatggtaaggccacctacgtgtcccgatacgtgcgaacttcccgactcaagcaggaggagttctttgagggagctaagtttatgaagattggagacctcaagggtctgttcggtctgttcactgtttacatgcagatgctccgagccaagctcaagattctcgacacctcctacggaaacggcactgctaacactgctctcgtgtaccaccacggtaagctcctggctctttctgaggctgataagccttacgctctcaaggttctggaggatggcgaccttcagacccttggtatgctcgactacgacaagcgactccttcactctttcactgcccaccctaaggttgaccctgtcactggtgagatgtttacctttggctacgctcacgagcccccttacatcacttaccgagttatttctaaggatggcattatgcaggaccctgtccccatcaccatccctgaggctatcatgatgcacgatttcgctattactgagaactacgctattatgatggacctccccctgtgtttccgacctaaggagatggtgaagaacaaccagcttgcttttaccttcgataccactaagaaggctcgatttggcgtcctccctcgatacgctaagtccgaggctcttatccgatggttcgagcttcccaactgcttcatttttcacaacgccaacgcttgggaggagggtgatgaggtcgtgctcattacctgccgacttccccaccctgacctcgacatggtcaacggagaggttaaggagaaccttgagaacttctccaacgagctgtacgagatgcgatttaacatgaagtctggtgccgcttctcagaagaagctctctgagtcttctgttgatttccctcgaatcaacgagaactacactggtcgaaagcagcgatacgtttacggcaccactctcaactctattgccaaggtcactggcatcatcaagttcgatctccacgccgagcctgagactggcaagaagcagcttgaggttggaggcaacgttcagggcatctttgaccttggtcccggtcgatttggttccgaggctgtctttgttccctctcagcccggaaccgagtgtgaggaggatgacggatacctgatcttcttcgttcacgatgagaacactggtaagtctgctgtgaacgtgattgatgccaagactatgtctgccgagcctgttgccgttgttgagcttcccaagcgagttccttacggattccacgccttctttgtcactgaggagcagattcaggagcaggccaagctgtaaa

SEQ ID NO: 12 - DNA, 9096 bp, synthetic construct
  source 1..9096: mol_type = other DNA
gttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccagatagtgcaatcacatgttgctactgtacctgctgtggaccacgcacggcggaacgtaccgtacaaatattttcttgctcacatgactctctctcggccgcgcacgccggtggcaaattgctcttgcattggctctgtctctagacgtccaaaccgtccaaagtggcagggtgacgtgatgcgacgcacgaaggagatggcccggtggcgaggaaccggacacggcgagccggcgggaaaaaaggcggaaaacgaaaagcgaagggcacaatctgacggtgcggctgccaccaacccaaggaggctattttgggtcgctttccatttcacattcgccctcaatggccactttgcggtggtgaacatggtttctgaaacaaccccccagaattagagtatattgatgtgtttaagattgggttgctatttggccattgtgggggagggtagcgacgtggaggacattccagggcgaattgagcctagaaagtggtaccattccaaccgtctcagtcgtccgaattgatcgctataactatcacctctctcacatgtctacttccccaaccaacatccccaacctcccccacactaaagttcacgccaataatgtaggcactctttctgggtgtgggacagcagagcaatacggaggggagattacacaacgagccacaattggggagatggtagccatctcactcgacccgtcgacttttggcaacgctcaattacccaccaaatttgggctggagttgaggggaccgtgttccagcgctgtaggaccagcaacacacacggtatcaacagcaaccaacgcccccgctaatgcacccagtactgcgcaggtgtgggccaggtgcgttccagatgcgagttggcgaaccctaagccgacagtgtactttttgggacgggcagtagcaatcgtgggcggagaccccggtgtatataaaggggtggagaggacggattattagcaccaacacacacacttatactacatgctagacacaaaaatggaccctaccggagacgttactgctagccctcgacctcaaaccaccattcctgtccgacaagcactctggggacttagccttgctggagccatcatcgccgcatgggtttttatgcacattggtttcgttttttttgccccccttgatcctatcgttctcgccctcgccccagttattattcttcttcaatcctggctttctgttggtctttttattatttctcacgacgcaattcacggttccctcgcccctggacgacccgcctttaatagagccatgggacgactctgcatgacactttacgccggtttcgactttgaccgtatggccgctgcacatcaccgacatcacagatcccctggaaccgccgctgaccccgatttttctgttgactcccctgatcgacctctcccttggtttggagctttcttccgacgttactttggctggagaccttttcttaccgttaacgctgtcgtctttacctactggcttgttcttggagctaaccctgttaatattgttctcttttatggcgttcctgcactcctttccgccggacagctcttttactttggtacatttctccctcaccgacacgaacgacaaggctttgctgatcaccaccgagcacgatccgtccgatccccttacatgctttctcttgttacttgttaccactttggaggctatcatcacgaacatcatctctttccacacgaaccctggtggcgcctgcctcaacgaggaggttgggaacgtgacagacgaaagagaaccggcccttaacgcgtgttttttgatcaatgatccaatggctttcacatacccccccacgcctataattaaaacacagagaaatataatctaacttaataaatattacggagaatctttcgagtgttcagcagaaatatagccattgtaacaaaagccggctatcgaccgctttatcgaagaatatttcccgccccccagtggccaaacgatatttaccgttcgtataatgtatgctatacgaagttataccggtctcgtagtgttcacgttcagttcacggtgagcttaaaactatcttcaagaagagatttgagacctgatttatacttgcagcaatgtttacttcttatcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgttaggggcagggcatgctcatgtagagcgcctgctcgccgtccgaggcggtgccgtcgtacagggcggtgtccaggccgcagagggtgaaccccatccgccggtacgcgtggatcgccggtgcgttgacgttggtgacctccagccagaggtgcccggcgccccgctcgcgggcgaactccgtcgcgagccccatcaacgcgcgcccgaccccgtgcccccggtgctccggggcgacctcgatgtcctcgacggtcagccggcggttccagccggagtacgagacgaccacgaagcccgccaggtcgccgtcgtccccgtacgcgacgaacgtccgggagtccgggtcgccgtcctccccgtcgtccgattcgtcgtccgattcgtcgtcggggaacaccttggtcaggggcgggtccaccggcacctcccgcagggtgaagccgtccccggtggcggtgacgcggaagacggtgtcggtggtgaaggacccatccagtgcctcgatggcctcggcgtcccccgggacactggtgcggtaccggtaagccgtgtcgtcaagagtggtacccatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccataacttcgtataatgtatgctatacgaacggtacccgggcaattctgcagatatcggaggctcttcctccacctccgaggtcggagactttgttgccaacaaggtcaaggagctgctcaagaaggagtaagtcgtttctacgacgcattgatggaaggagcaaactgacgcgcctgcgggttggtctaccggcagggtccgctagtgtataagactctataaaaagggcccagccctgctaatgaaatgatgatttataatttaccggtgtagcaaccttgactagaagaagcagattgggtgtgtttgtagtggaggacagtggagccgaattccagcacactggcggccgttactagtttgccacctacaagccagattttctatttacaacaatatctggtcaaatttcagtttcgttacataaatcgttatgtcaaaggagtgtgggaggttaagagaattatcaccggcaaactatctgttaattgcacgcgttaagggccggttctctttcgtctgtcacgttcccaacctcctcgttgaggcaggcgccaccagggttcgtgtggaaagagatgatgttcgtgatgatagcctccaaagtggtaacaagtaacaagagaaagcatgtaaggggatcggacggatcgtgctcggtggtgatcagcaaagccttgtcgttcgtgtcggtgagggagaaatgtaccaaagtaaaagagctgtccggcggaaaggagtgcaggaacgccataaaagagaacaatattaacagggttagctccaagaacaagccagtaggtaaagacgacagcgttaacggtaagaaaaggtctccagccaaagtaacgtcggaagaaagctccaaaccaagggagaggtcgatcaggggagtcaacagaaaaatcggggtcagcggcggttccaggggatctgtgatgtcggtgatgtgcagcggccatacggtcaaagtcgaaaccggcgtaaagtgtcatgcagagtcgtcccatggctctattaaaggcgggtcgtccaggggcgagggaaccgtgaattgcgtcgtgagaaataataaaaagaccaacagaaagccaggattgaagaagaataataactggggcgagggcgagaacgataggatcaaggggggcaaaaaaaacgaaaccaatgtgcataaaaacccatgcggcgatgatggctccagcaaggctaagtccccagagtgcttgtcggacaggaatggtggtttgaggtcgagggctagcagtaacgtctccggtagggtccatttttgtgtctagcgaatgattcttatactcagaaggaaatgcttaacgatttcgggtgtgagttgacaaggagagagagaaaagaagaggaaaggtaattcggggacggtggtcttttatacccttggctaaagtcccaaccacaaagcaaaaaaattttcagtagtctattttgcgtccggcatgggttacccggatggccagacaaagaaactagtacaaagtctgaacaagcgtagattccagactgcagtaccctacgcccttaacggcaagtgtgggaaccgggggaggtttgatatgtggggtgaagggggctctcgccggggttgggcccgctactgggtcaatttggggtcaattggggcaattggggctgttttttgggacacaaatacgccgccaacccgtctctggtaccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacat

SEQ ID NO: 13 - DNA, 8809 bp, synthetic construct
  source 1..8809: mol_type = other DNA
gttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccagagacgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcgctagccacaaaaatgctgtcgaagctgcagtcaatcagcgtcaaggcccgccgcgttgaactcgcccgcgacatcacgcggcccaaagtctgcctgcatgctcagcggtgctcgctcgttcggctgcgagtggcagcaccacagacagaggaggcggtgggaaccgtgcaggctgccggcgcgggcgatgagcacagcgccgatgttgcactccagcagcttgaccgggctatcgcagagcgtcgtgcccggcgcaaacgggagcagctctcataccaggctgccgccattgcagcatcaattggcgtgtcaggcattgccatcttcgccacctacctgagatttgccatgcacatgaccgtgggcggagcagtgccatggggtgaagtggctggcactctcctcttggtggttggtggcgcgctcggcatggagatgtatgcccgctatgcccacaaagccatctggcatgagtcgcctctgggctggctgctgcacaagagccaccacacacctcgcactggaccctttgaagccaacgacttgtttgcaatcatcaatggactgcccgccatgctcctgtgtacctttggcttctggctgcccaacgtcttgggtgcggcctgctttggcgcgggtctgggcatcacgctctacggcatggcatatatgtttgttcacgatggcctggtgcacagacgctttcccaccggacccatcgctggcctgccctacatgaagcgcctgacagtggcccaccagcttcaccacagcggcaagtacggtggcgcgccctggggcatgttcttgggtccacaggagctgcagcacattccaggtgctgcggaggaggtggagcgactcgtcctggaactggactggtcaaagcggtaaacgcgtgcaattaacagatagtttgccggtgataattctcttaacctcccacactcctttgacataacgatttatgtaacgaaactgaaatttgaccagatattgttgtaaatagaaaatctggcttgtaggtggcaaactagtaacggccgccagtgtgctggaattgaatatttaccgttcgtataatgtatgctatacgaagttataccggtctcgtagtgttcacgttcagttcacggtgagcttaaaactatcttcaagaagagatttgagacctgatttatacttgcagcaatgtttacttcttatcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgtctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatgggtacctgagaacatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccataacttcgtataatgtatgctatacgaacggtacccgggcaattctgcagatattcaattccagcacactggcggccgttactagtttgccacctacaagccagattttctatttacaacaatatctggtcaaatttcagtttcgttacataaatcgttatgtcaaaggagtgtgggaggttaagagaattatcaccggcaaactatctgttaattgcacgcgtttaccgctttgaccagtccagttccaggacgagtcgctccacctcctccgcagcacctggaatgtgctgcagctcctgtggacccaagaacatgccccagggcgcgccaccgtacttgccgctgtggtgaagctggtgggccactgtcaggcgcttcatgtagggcaggccagcgatgggtccggtgggaaagcgtctgtgcaccaggccatcgtgaacaaacatatatgccatgccgtagagcgtgatgcccagacccgcgccaaagcaggccgcacccaagacgttgggcagccagaagccaaaggtacacaggagcatggcgggcagtccattgatgattgcaaacaagtcgttggcttcaaagggtccagtgcgaggtgtgtggtggctcttgtgcagcagccagcccagaggcgactcatgccagatggctttgtgggcatagcgggcatacatctccatgccgagcgcgccaccaaccaccaagaggagagtgccagccacttcaccccatggcactgctccgcccacggtcatgtgcatggcaaatctcaggtaggtggcgaagatggcaatgcctgacacgccaattgatgctgcaatggcggcagcctggtatgagagctgctcccgtttgcgccgggcacgacgctctgcgatagcccggtcaagctgctggagtgcaacatcggcgctgtgctcatcgcccgcgccggcagcctgcacggttcccaccgcctcctctgtctgtggtgctgccactcgcagccgaacgagcgagcaccgctgagcatgcaggcagactttgggccgcgtgatgtcgcgggcgagttcaacgcggcgggccttgacgctgattgactgcagcttcgacagcatttttgtggctagcgaatgattcttatactcagaaggaaatgcttaacgatttcgggtgtgagttgacaaggagagagagaaaagaagaggaaaggtaattcggggacggtggtcttttatacccttggctaaagtcccaaccacaaagcaaaaaaattttcagtagtctattttgcgtccggcatgggttacccggatggccagacaaagaaactagtacaaagtctgaacaagcgtagattccagactgcagtaccctacgcccttaacggcaagtgtgggaaccgggggaggtttgatatgtggggtgaagggggctctcgccggggttgggcccgctactgggtcaatttggggtcaattggggcaattggggctgttttttgggacacaaatacgccgccaacccgtctctggtaccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacat

SEQ ID NO: 14 - DNA, 12810 bp, synthetic construct
  source 1..12810: mol_type = other DNA
ggttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctggcctaggaagcgacttccaatcgctttgcatatccagtaccacacccacaggcgtttgtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaacaatggacaagaagtactccatcggtttggacattggtactaactctgtcggctgggccgtcatcaccgacgagtacaaggttccctccaagaagttcaaggtccttggcaacaccgaccgacactctatcaagaagaacctgatcggtgctctgctgttcgactctggcgagactgccgaggccacccgactgaagcgaaccgctcgacgccgatacacccgacgaaagaaccgaatctgttacctccaggagatcttcagcaacgagatggctaaggtcgacgactccttcttccaccgactcgaggagtctttcctggtcgaagaggataagaagcacgagcgacaccccatcttcggcaacattgttgatgaggttgcctaccatgagaagtaccccaccatctaccacctccgaaagaagctcgtcgactccactgacaaggctgacctccgactcatctaccttgctctcgcccacatgatcaagttccgaggtcacttcctcattgagggtgatctcaaccccgacaactccgacgttgacaagctgttcatccagctcgtccagacctacaaccagctctttgaggagaaccctatcaacgcttctggtgttgacgccaaggccattctctccgcccgactctctaagtcccgacgactcgagaacctcattgcccagctgcccggcgagaagaagaacggcctcttcggtaacctgattgctctctctcttggtctgacccccaacttcaagtccaactttgacctcgccgaggacgccaagctccagctgtccaaggacacctacgatgacgatctggacaacctcctggcccagatcggtgaccagtacgccgatctcttccttgccgccaagaacctctccgacgccatcctgctctccgacatcctccgagtcaacaccgagattaccaaggctcctctgtctgcctctatgatcaagcgatacgacgagcaccaccaggatctcactcttctcaaggctctcgtccgacagcagctccccgagaagtacaaggagattttctttgaccagtccaagaacggttacgctggctacattgacggtggtgcttcccaggaagagttttacaagttcatcaagcctattctggagaagatggacggtaccgaggagctgctcgtcaagctcaaccgagaggacctccttcgaaagcagcgaaccttcgataacggctccatcccccaccagatccacctgggtgagctccacgccattctccgaagacaagaggacttctaccccttcctaaaggataaccgagagaagatcgagaagattctcaccttccgaatcccctactacgtcggtcccctcgctcgaggtaactcccgatttgcttggatgacccgaaagtccgaggagactatcaccccctggaactttgaagaggtagtcgacaagggtgcctccgcccagtctttcattgagcggatgaccaacttcgataagaacctccccaacgagaaggtccttcccaagcactctctcctctacgagtacttcaccgtctacaacgagctgaccaaggtcaagtacgttaccgagggcatgcgaaagcccgctttcctctctggtgagcagaagaaggccattgtcgacctcctgttcaagactaaccgaaaagtcaccgtcaagcagctcaaggaagactacttcaagaagattgagtgcttcgactccgtcgagatttccggtgtcgaggaccgattcaacgcctccctcggcacctaccacgatcttctgaagatcatcaaggacaaggactttcttgataacgaggagaacgaggacattctcgaggacatcgtcctcaccctcacccttttcgaggatcgagagatgatcgaggagcgactcaagacctacgcccatctcttcgacgacaaggtcatgaagcaactcaagcgacgacgatacactggctggggccgactttcccgaaagctcatcaacggcatccgagacaagcagtctggcaagaccatcctggacttcctgaagtccgacggtttcgccaaccgaaacttcatgcagctcatccacgacgactctcttaccttcaaagaggatatccagaaggcccaggtttctggccagggcgactccctccacgagcacattgccaacctcgccggatcccccgccatcaaaaagggtatcctccagaccgtcaaggttgtcgacgaactcgtgaaggtcatgggccgacacaagcccgagaacatcgttatcgagatggcccgagagaaccagaccacccagaagggtcagaagaactcccgagagcgaatgaagcgaatcgaagagggtatcaaggagctcggttcccagattctcaaggagcaccccgtcgagaacacccagctccagaacgagaaactctacctgtactacctccagaatggccgagacatgtacgttgaccaggagctcgacatcaaccgactctccgactacgacgtcgaccacattgttcctcagtccttcctcaaggacgactccatcgacaacaaggttctgacccgatctgacaagaaccgaggtaagtccgacaacgttccctccgaagaggtcgttaagaagatgaagaactactggcgacagcttctcaacgccaaactgatcacccagcgaaagtttgacaacctcaccaaggccgagcgaggtggtctgtccgagctggacaaggccggcttcattaagcgacagctggtcgagactcgacagatcaccaagcacgtcgcccagatcctcgactcccgaatgaacaccaagtacgacgagaacgacaagctcatccgggaggtcaaggtcatcaccctgaagtctaagcttgtctccgacttccgaaaggacttccagttctacaaggtccgagagatcaacaactaccaccacgcccacgacgcctacctcaacgccgttgttggtaccgccctcatcaagaagtatcccaagctcgagtccgagttcgtttacggcgactacaaggtttacgatgtccgaaagatgattgccaagtccgagcaggagatcggtaaggccaccgccaagtactttttctactccaacatcatgaatttcttcaagaccgagatcactctcgccaacggtgagattcgaaagcgacccctgattgagactaatggtgagactggtgagatcgtctgggataagggccgagacttcgccaccgtccgaaaggtcctgtccatgccccaggtcaacattgtcaagaagaccgaggtccagaccggtggcttctccaaggagtccattctccccaagcgaaactccgacaaactcatcgcccgtaagaaggactgggatccgaagaagtacggtggtttcgattctcccaccgttgcctactccgtcctcgttgttgctaaagtcgagaagggtaagtctaagaaactcaagtccgtgaaggagctactcggtatcaccatcatggagcgatcttcttttgagaagaaccccattgacttcctcgaggccaagggttacaaagaggtcaagaaggacctgattatcaagctgcccaagtactccctctttgagctcgagaacggccgaaagcgaatgctggcttccgctggtgagctgcagaagggcaacgagctcgctctgccctccaagtacgtcaacttcctctacctggcctcccactacgagaagctcaagggctcccccgaggacaacgagcagaagcagctgttcgttgagcagcacaagcactacctcgacgagatcatcgagcagatctccgagttctccaagcgagtcatcctcgctgacgccaaccttgataaggttctctctgcttacaacaagcaccgggacaagcccatccgagagcaggccgagaatatcatccacctcttcactctcaccaacctcggcgctcctgctgccttcaagtacttcgacaccaccattgaccgaaagaggtacacctccaccaaggaagtcctcgacgccaccctgatccaccagtccatcaccggcctctacgaaacccgaatcgacctctcccagctcggcggtgactctcgagccgaccccaagaagaagcgaaaagtctaaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattattacctcttgcccatcgaacgtacaagtactcctctgttctctccttcctttgctttgtgcacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacaatgtgtctactgatgagtccgtgaggacgaaacgagtaagctcgtctagacagcaggcgcatcgtagttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaacttcgagctaatccagtagcttacgttacccaggggcaggtcaactggctagccacgagtctgtcccaggtcgcaatttagtgtaataaacaatatatatattgagtctaaagggaattgtagctattgtgattgtgtgattttcgtcttgctggttcttattgtgtcccattcgtttcatcctgatgaggacccctggaaccggtgttttcttagtctctgcaatcgctagtcttgttgctatgacagttgcgtcgacactattcaggtcatctatcggttattctgatattataatacctccggatcgatgtacctgatttatacttgcagcaatgtttacttcttatcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgtctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatgggtacctgagaacatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccgaattttcagaacctaatttatctgttacccggcctgtggctcgcacagcttaaaaatggtcaaactttccccttcttgtctttttttcctcacattcatcaggttcttgtcttgatctttcaagtgagtattaattaccgaccttggttcttcattgggagagcattggaagccgtggtgcagcaaccacaaaacggttcttccccttcgataccttcttgcctgcctttcaatacaagtcggctcgattagcggtggtcgcccccgccagcggagaacatggaactaacccagaatgagagctaagtggagaaagaagagagtcagacgactcaagcgaaagcgccgcaaggtccgagctcgatccaaataagcggtttttaacggagatttaacactaaatcgaagaacttttcccgtttcatttgcgaatgagctcgttaacaaaatcccccagtttttttatccagctgtaaggattgacattagtaatgaattattgtttggtatatttaaatctgtagttcctttctgtccgtgtcggcaactgtcgtactcgtgatttacttgtattgacgaatacttactgtagcgcactctgctgctactggtcgtaaggatgtgctatttcggtgtatggtgggttttttgggggtcggaaccgaagactgttacacgggcacggctcgttgtgtacacgcacagagctcttgcgagtcatgttgtagctagctcgtcgtgttcaggaactgttcgatggttcggagagagtcgccgcccagaacatacgcgcaccgatgtcagcagacagccttattacaagtatattcaagcaagtatatccgtagggtgcgggtgatttggatctaaggttcgtactcaacactcacgagcagcttgcctatgttacatccttttatcagacataacataattggagtttacttacacacggggtgtacctgtatgagcaccacctacaattgtagcactggtacttgtacaaagaatttattcgtacgaatcacagggacggccgccctcaccgaaccagcgaatacctcagcggtcccctgcagtgactcaacaaagcgatatgaacatcttgcgatggtatcctgctgatagtttttactgtacaaacacctgtgtagctccttctagcatttttaagttattcacacctcaaggggagggataaattaaataaattccaaaagcgaagatcgagaaactaaattaaaattccaaaaacgaagttggaacacaaccccccgaaaaaaaacaacaaacaaaaaacccaacaaaataaacaaaaacaaaataaatatataactaccagtatctgactaaaagttcaaatactcgtacttacaacaaatagaaatgagccggccaaaattctgcagaaaaaaatttcaaacaagtactggtataattaaattaaaaaacacatcaaagtatcataacgttagttattttattttatttaataaaagaaaacaacaagatgggctcaaaactttcaacttatacgatacataccaaataacaatttagtatttatctaagtgcttttcgtagataatggaatacaaatggatatccagagtatacacatggatagtatacactgacacgacaattctgtatctctttatgttaactactgtgaggcattaaatagagcttgatatataaaatgttacatttcacagtctgaacttttgcagattacctaatttggtaagatattaattatgaactgaaagttgatggcatccctaaatttgatgaaagatgaaattgtaaatgaggtggtaaaagagctacagtcgttttgttttgagataccatcatctctaacgaaatatctattaaaaatctcagtgtgatcatgagtcattgccatcctggaaaatgtcatcatggctgatatttctaactgtttacttgagataaatatatatttacaagaacttcccttgaaattaatttagatataaaatgtttgcgggcaagttactacgaggaataaattatatctaga

(19) United States
(12) Patent Application Publication
(10) Pub. No.: US 2026/0226430 A1
(43) Pub. Date: Aug. 6, 2026

(54) CARRP ENZYME VARIANTS AND THEIR USE IN PRODUCING CAROTENOID AND APOCAROTENOID

(71) Applicant: DSM IP ASSETS B.V., TE HEERLEN (NL)

(72) Inventors: Valmik Kanubhai VYAS, Winchester, MA (US); René Marcel DE JONG, Amsterdam (NL); René VERWAAL, Den Haag (NL); Peter Louis HOUSTON, Boston, MA (US); Maria Elena MAYORGA, Medford, MA (US); John ROYER, Lexington, MA (US); Anna SYMBOR-NAGRABSKA, Reading, MA (US)

(21) Appl. No.: 19/150,719

(22) PCT Filed: Jan. 29, 2024
(86) PCT No.: PCT/EP2024/052032

§ 371 Date: Jul. 24, 2025

Related U.S. Application Data
(60) Provisional application No. 63/482,169, filed on Jan. 30, 2023.

(30) Foreign Application Priority Data
Aug. 1, 2023 (EP) 23188889.2

(51) Int. Cl. C12N 9/10; C12N 9/90; C12P 5/00; C12P 23/00

(52) U.S. Cl. CPC C12N 9/1085; C12N 9/90; C12P 5/007; C12P 23/00; C12Y 205/01032; C12Y 505/01019

(57) ABSTRACT

The present invention is related to increased accumulation of carotenoids and derivatives thereof via manipulation and heterologous expression of CarRP derived from Mucor circinelloides.

The present invention is related to increased accumulation of carotenoids and derivatives thereof via manipulation and heterologous expression of CarRP derived from Mucor circinelloides.

Carotenoids, including C-40 isoprenoid compounds such as carotenes and xanthophylls as well a as cleavage products such as apocarotenoids, are responsible for the orange color of carrots, as well as the pink in flamingos and salmon, the red in lobsters or shrimp and for further important applications in the food, feed, cosmetic or pharmaceutical industry. Moreover, beta-carotene is a key precursor or intermediate in synthesis of vitamin A.

Retinoids, belonging to the class of apocarotenoids, are one of very important and indispensable nutrient factors for both human and animals which must be supplied via diet. Retinoids promote well-being of humans/animals, inter alia in respect of vision, the immune system and growth.

Since chemical synthesis of carotenoids or retinoids has some major disadvantages, i.e. consumption of energy and/or water, organic and/or inorganic solvents, synthesis of undesired side products, and the rising worldwide demand for natural products to be used as e.g. colorants or nutritional supplements, there is a strong need for biotechnological production of such compounds.

Carotenoids including carotenes and xanthophylls as well as apocarotenoids including retinoids and ionones are naturally produced by certain organisms, including photosynthetic organisms (e.g., plants, algae, cyanobacteria—especially with regards to production of carotenoids, and some fungi, such as e.g. Mucor circinelloides, Yarrowia, Saccharomyces (both for production of carotenoids and retinoids) as well as in bacteria, such as e.g. E. coli or Paraccocus. However, these systems are industrially intractable and/or produce the compounds at such low levels that commercial scale isolation is not practicable.

A key-enzyme in both carotenoid and apocarotenoid biosynthesis is the bifunctional enzyme CarRP catalyzing on the one hand the conversion of geranylgeranyl pyrophosphate (GGPP) into phytoene, i.e. acting as phytoene synthase, and additionally conversion of lycopene into beta-carotene, i.e. acting as lycopene cyclase. A widely used enzyme with good performance is originated from Mucor circinelloides (McCarRP), however, accumulation of lycopene can lead to feedback inhibition of CarRP and thus reduced production of beta-carotene and xanthophylls.

Thus, there is a strong need for more efficient bio-production of carotenoids and apocarotenoids, including but not limited to retinal or retinol, wherein the respective genes are (over) expressed in generally recognized as safe (GRAS) host cells such as e.g. oleagenous yeast, with reduction or elimination of known bottlenecks in such production processes.

Surprisingly, we now have identified amino acid residues in CarRP originated from Mucor circinelloides (McCarRP) that are critical for formation of phytoene and/or beta-carotene, and thus critical for production of carotenoids or apocarotenoids, whereby known feedback inhibition is reduced. Introduction of one or more amino acid substitution(s) located in both the lycopene cyclase (R)- and the phytoene synthase (P)-domain of said enzyme leads to increased formation of said carotenoid compounds in the range of at least about 5%, such as e.g. in a range of 20-75% and more, as compared to the wild-type (non-modified) McCarRP according to SEQ ID NO:1 or shown in FIG. 1.

Particularly, the present invention is related to a modified bifunctional enzyme involved in synthesis of phytoene and acting as lycopene cyclase as well as to a method for generation of such modified enzyme, i.e. enzyme catalyzing the conversion of geranylgeranyl pyrophosphate (GGPP) into phytoene and/or conversion of lycopene into beta-carotene, particularly CarRP comprising one or more modification(s), such as amino acid substitution(s) being introduced into a sequence with at least about 20%, such as 25, 30, 35, 40, 45, 50, 55, 60, 65, 70, 75, 80, 85, 90, 92, 95, 97, 98, 99% or up to 100% identity to SEQ ID NO:1 or shown in FIG. 1, said one or more amino acid substitution(s) being introduced at position(s) corresponding to amino acid residue(s) selected from the group consisting of position 7, 33, 153, 159, 167, 194, 305, 330, 430, 431, 432, 476, 547, 579, and combinations thereof, in the polypeptide according to SEQ ID NO:1 or shown in FIG. 1.

More particularly, the present invention is directed to modified CarRP as defined herein comprising one or more amino acid substitution(s) on position(s) as defined herein, wherein the amino acid residue on position corresponding to position 7 in SEQ ID NO:1 or shown in FIG. 1 is different from glutamic acid or glutamine, wherein the amino acid residue on position corresponding to position 33, in SEQ ID NO:1 or shown in FIG. 1 is different from alanine or tryptophane, wherein the amino acid residue on position corresponding to position 153 in SEQ ID NO:1 or shown in FIG. 1 is different from alanine, wherein the amino acid residue on position corresponding to position 159 in SEQ ID NO: 1 or shown in FIG. 1 is different from leucine, wherein the amino acid residue on position corresponding to position 167 in SEQ ID NO:1 or shown in FIG. 1 is different from tyrosine, wherein the amino acid residue on position corresponding to position 194 or 476 in SEQ ID NO:1 or shown in FIG. 1 is different from isoleucine, wherein the amino acid residue on position corresponding to position 305 in SEQ ID NO:1 or shown in FIG. 1 is different from threonine, wherein the amino acid residue on position corresponding to position 330 in SEQ ID NO:1 or shown in FIG. 1 is different from aspartic acid, wherein the amino acid residue on position corresponding to position 430 or 431 in SEQ ID NO:1 or shown in FIG. 1 is different from serine, wherein the amino acid residue on position corresponding to position 432 or 547 in SEQ ID NO:1 or shown in FIG. 1 is different from valine, and/or wherein the amino acid residue on position corresponding to position 579 in SEQ ID NO:1 or shown in FIG. 1 is different from arginine.

In some embodiments, the use of such modified enzyme in a process for production of carotenoids and apocarotenoids, including carotenes, xanthophylls, retinoids and ionones, wherein said modified enzyme is expressed, particularly heterologous expressed, in a suitable host cell, particularly fungal carotenoid and/or retinoid producing host cell, more particularly a beta-carotene producing host cell, leads to an increase in the titer of such products in the range of at least about 5%, such as e.g. 5 to 20% or even 5 to 75%, based on total carotenoids/apocarotenoids/retinoids present in/produced by said modified host cell as compared to the process using the same conditions but a non-modified CarRP enzyme according to SEQ ID NO:1 or shown in FIG. 1.

The terms “CarRP”, “phytoene synthase”, “lycopene cyclase”, “CrtYB” are used interchangeably herein and refer to bi-functional enzymes involved in the biosynthetic pathway from GGPP into beta-carotene which are capable of catalyzing the conversion of GGPP into phytoene, i.e. functioning as phytoene synthase [EC 2.5.1.32], and/or conversion of lycopene into beta-carotene, i.e. functioning as lycopene beta-cyclase [EC 5.5.1.19]. An example and suitable enzyme which can be used for generation of the modified enzymes according to the present invention is the McCarRP as shown in SEQ ID NO:1 or shown in FIG. 1 or enzymes with at least about 20% identity to SEQ ID NO:1 or shown in FIG. 1, including enzymes encoded by a polynucleotide according to SEQ ID NO:2.

A “modified” CarRP as defined herein based on a non-modified CarRP, particularly based on an enzyme with at least about 20% identity to McCarRP according to SEQ ID NO:1 or shown in FIG. 1, shows increased enzyme activity, i.e. increased activity towards formation of phytoene and/or beta-carotene and thus increase in the formation of apocarotenoids, including retinoids, and/or carotenoids including xanthophylls as defined herein, particularly increase by at least about 5%, such as in the range of 5 to 75%, particularly 20 to 75% and more (especially with regards to retinoid production) as compared to formation of said products using an enzyme according to SEQ ID NO:1 or shown in FIG. 1.

The term “carotenoids” as used herein is well known in the art. It includes long, 40 carbon conjugated isoprenoid polyenes (C-40 isoprenoids) that are formed in nature by the ligation of two 20 carbon GGPP molecules. These include but are not limited to phytoene, lycopene, beta-carotene, alpha-carotene, gamma-carotene, rhodoxanthin, canthaxanthin, zeaxanthin, astaxanthin, beta-cryptoxanthin or lutein. Biosynthesis of carotenoids is described in e.g. WO2006102342. The term carotenoids also includes the group of “xanthophylls”, i.e. oxidized carotenoid derivatives such as e.g. lutein, zeaxanthin or beta-cryptoxanthin.

As used herein, “apocarotenoids” are cleavage products of carotenoids and thus defined as <C40-carotenoids and include but are not limited to retinoids or ionones, such as e.g. retinal, retinol, retinyl acetate, beta-ionone or alpha-ionone.

Retinoids as used herein include but are not limited to retinal, retinolic acid, retinol, retinoic methoxide, retinyl acetate, retinyl esters, 4-keto-retinoids, 3 hydroxy-retinoids or combinations thereof. Long chain retinyl esters as used herein are defined as hydrocarbon esters of retinol with fatty acids, where the fatty acids consist of at least about 8, such as e.g. 9, 10, 12, 13, 15 or 20 carbon atoms and up to about 26, such as e.g. 25, 22, 21 or less carbon atoms, with preferably up to about 6 unsaturated bonds, such as e.g. 0, 1, 2, 4, 5, 6 unsaturated bonds. The fatty acids in the long chain retinyl esters include but are not limited to linoleic acid, oleic acid or palmitic acid. Biosynthesis of retinoids is described in e.g. WO2008042338 or WO2019058000, wherein enzymatic conversion of beta-carotene into retinal into retinol into retinyl acetate in a strain of Yarrowia lypolitica expressing the respective heterologous genes is disclosed.

The terms “conversion”, “enzymatic conversion” in connection with enzymatic catalysis of GGPP and/or lycopene are used interchangeably herein and refer to the action of modified or non-modified CarRP used as biocatalyst in conversion of GGPP into phytoene or lycopene into beta-carotene as defined herein, thus including synthase or cyclase activities of CarRP as described herein.

Suitable host cells according to the present invention include fungal host cells. As used herein, the term “fungal host cell” particularly includes GRAS host cells such as particularly yeast cells, wherein the cell is a carotenoid and/or apocarotenoid-producing host cell, particularly a beta-carotene and/or retinol producing fungal host cell, including but not limited to Yarrowia or Saccharomyces, such as e.g. Yarrowia lipolytica or Saccharomyces cerevisiae.

The modified enzyme might be used in an isolated form (e.g. in a cell-free system) or might be expressed in the suitable host cell, such as e.g. carotenoid and/or apocarotenoid-producing host cell, particularly fungal host cell as defined herein. Enzymes might be expressed as endogenous enzymes or as heterologous enzymes. Preferably, the modified enzymes as described herein are introduced and expressed as heterologous enzymes in a suitable host cell, such as e.g. a carotenoid and/or apocarotenoid-producing host cell, preferably carotene and/or retinol-producing host cell, particularly fungal host cell as defined herein.

In one embodiment, the modified CarRP enzyme as defined herein comprises an amino acid substitution at a position corresponding to residue 7 in the polypeptide according to SEQ ID NO:1 or shown in FIG. 1, particularly introduction of aspartic acid, e.g. via substitution of glutamic acid (E7D), wherein the modified enzyme is derived from an enzyme with at least about 20% identity to McCarRP according to SEQ ID NO:1 or shown in FIG. 1. Using such modified enzyme comprising said mutation in a fermentation process, wherein said modified enzyme is introduced and expressed under suitable conditions using a carotenoid or apocarotenoid-producing host cell as specified herein, the formation of carotenoids could be increased by at least about 5-30%, such as e.g. 10, 15, 20, 25, 30% or more, the formation of apocarotenoids, particularly retinoids, could be increased by at least about 5-67%, such as e.g. 10, 15, 20, 25, 30, 35, 40, 45, 50, 55, 60, 65, 70% or more, as compared to the corresponding process using the non-modified CarRP according to SEQ ID NO:1 or shown in FIG. 1, including McCarRP according to SEQ ID NO:1 or shown in FIG. 1 but with glutamine on a position 7 in the polypeptide according to SEQ ID NO:1 or shown in FIG. 1.

In one embodiment, the modified CarRP enzyme as defined herein comprises an amino acid substitution at a position corresponding to residue 33 in the polypeptide according to SEQ ID NO:1 or shown in FIG. 1, particularly introduction of asparagine, e.g. via substitution of alanine (A33N), wherein the modified enzyme is derived from an enzyme with at least about 20% identity to McCarRP according to SEQ ID NO:1 or shown in FIG. 1. Using such modified enzyme comprising said mutation in a fermentation process, wherein said modified enzyme is introduced and expressed under suitable conditions using a carotenoid or apocarotenoid-producing host cell as specified herein, the formation of carotenoids could be increased by at least about 5-30%, such as e.g. 10, 15, 20, 25, 30% or more, the formation of apocarotenoids, particularly retinoids, could be increased by at least about 20-46%, such as e.g. 25, 30, 35, 40, 45, 50% or more as compared to the corresponding process using the non-modified CarRP according to SEQ ID NO:1 or shown in FIG. 1, including McCarRP according to SEQ ID NO:1 or shown in FIG. 1 but with tryptophane on a position 33 in the polypeptide according to SEQ ID NO:1 or shown in FIG. 1.

In some embodiment, the modified CarRP enzyme as defined herein comprises an amino acid substitution at a position corresponding to residue 153 in the polypeptide according to SEQ ID NO:1 or shown in FIG. 1, particularly introduction of serine, e.g. via substitution of alanine (A153S), wherein the modified enzyme is derived from an enzyme with at least about 20% identity to McCarRP according to SEQ ID NO:1 or shown in FIG. 1. Using such modified enzyme comprising said mutation in a fermentation process, wherein said modified enzyme is introduced and expressed under suitable conditions using a carotenoid or apocarotenoid-producing host cell as specified herein, the formation of carotenoids could be increased by at least about 5-30%, such as e.g. 10, 15, 20, 25, 30% or more, the formation of apocarotenoids, particularly retinoids, could be increased by at least about 20-43%, such as e.g. 25, 30, 35, 40, 45% or more, as compared to the corresponding process using the non-modified CarRP according to SEQ ID NO:1 or shown in FIG. 1.

In some embodiment, the modified CarRP enzyme as defined herein comprises an amino acid substitution at a position corresponding to residue 159 in the polypeptide according to SEQ ID NO:1 or shown in FIG. 1, particularly introduction of valine, e.g. via substitution of leucine (L159V), wherein the modified enzyme is derived from an enzyme with at least about 20% identity to McCarRP according to SEQ ID NO:1 or shown in FIG. 1. Using such modified enzyme comprising said mutation in a fermentation process, wherein said modified enzyme is introduced and expressed under suitable conditions using a carotenoid or apocarotenoid-producing host cell as specified herein, the formation of carotenoids could be increased by at least about 5-30%, such as e.g. 10, 15, 20, 25, 30% or more, the formation of apocarotenoids, particularly retinoids, could be increased by at least about 20-41%, such as e.g. 25, 30, 35, 40, 45% or more, as compared to the corresponding process using the non-modified CarRP according to SEQ ID NO:1 or shown in FIG. 1.

In one embodiment, the modified CarRP enzyme as defined herein comprises an amino acid substitution at a position corresponding to residue 167 in the polypeptide according to SEQ ID NO:1 or shown in FIG. 1, particularly introduction of phenylalanine, e.g. via substitution of tyrosine (Y167F), wherein the modified enzyme is derived from an enzyme with at least about 20% identity to McCarRP according to SEQ ID NO:1 or shown in FIG. 1. Using such modified enzyme comprising said mutation in a fermentation process, wherein said modified enzyme is introduced and expressed under suitable conditions using a carotenoid or apocarotenoid-producing host cell as specified herein, the formation of carotenoids could be increased by at least about 5-30%, such as e.g. 10, 15, 20, 25, 30% or more, the formation of apocarotenoids, particularly retinoids, could be increased by at least about 20-24%, as compared to the corresponding process using the non-modified CarRP according to SEQ ID NO:1 or shown in FIG. 1.

In one embodiment, the modified CarRP enzyme as defined herein comprises an amino acid substitution at a position corresponding to residue 194 in the polypeptide according to SEQ ID NO:1 or shown in FIG. 1, particularly introduction of leucine, e.g. via substitution of isoleucine (I194L), wherein the modified enzyme is derived from an enzyme with at least about 20% identity to McCarRP according to SEQ ID NO:1 or shown in FIG. 1. Using such modified enzyme comprising said mutation in a fermentation process, wherein said modified enzyme is introduced and expressed under suitable conditions using a carotenoid or apocarotenoid-producing host cell as specified herein, the formation of carotenoids could be increased by at least about 5-30%, such as e.g. 10, 15, 20, 25, 30% or more, the formation of apocarotenoids, particularly retinoids, could be increased by at least about 20-35%, such as e.g. 25, 30, 35, 40% or more, as compared to the corresponding process using the non-modified CarRP according to SEQ ID NO:1 or shown in FIG. 1.

In one embodiment, the modified CarRP enzyme as defined herein comprises an amino acid substitution at a position corresponding to residue 305 in the polypeptide according to SEQ ID NO:1 or shown in FIG. 1, particularly introduction of alanine, e.g. via substitution of threonine (T305A), wherein the modified enzyme is derived from an enzyme with at least about 20% identity to McCarRP according to SEQ ID NO:1 or shown in FIG. 1. Using such modified enzyme comprising said mutation in a fermentation process, wherein said modified enzyme is introduced and expressed under suitable conditions using a carotenoid or apocarotenoid-producing host cell as specified herein, the formation of carotenoids could be increased by at least about 5-30%, the formation of apocarotenoids, particularly retinoids, could be increased by at least about 20-23%, as compared to the corresponding process using the non-modified CarRP according to SEQ ID NO:1 or shown in FIG. 1.

In one embodiment, the modified CarRP enzyme as defined herein comprises an amino acid substitution at a position corresponding to residue 330 in the polypeptide according to SEQ ID NO:1 or shown in FIG. 1, particularly introduction of glutamic acid or asparagine, e.g. via substitution of aspartic acid (D330E or D330N), wherein the modified enzyme is derived from an enzyme with at least about 20% identity to McCarRP according to SEQ ID NO:1 or shown in FIG. 1. Using such modified enzyme comprising said mutation in a fermentation process, wherein said modified enzyme is introduced and expressed under suitable conditions using a carotenoid or apocarotenoid-producing host cell as specified herein, the formation of carotenoids could be increased by at least about 5-30%, the formation of apocarotenoids, particularly retinoids, could be increased by at least about 5-27%, such as e.g. 10, 15, 20, 25, 30% or more, as compared to the corresponding process using the non-modified CarRP according to SEQ ID NO:1 or shown in FIG. 1.

In one embodiment, the modified CarRP enzyme as defined herein comprises an amino acid substitution at a position corresponding to residue 430 in the polypeptide according to SEQ ID NO:1 or shown in FIG. 1, particularly introduction of alanine, e.g. via substitution of serine (S430A), wherein the modified enzyme is derived from an enzyme with at least about 20% identity to McCarRP according to SEQ ID NO:1 or shown in FIG. 1. Using such modified enzyme comprising said mutation in a fermentation process, wherein said modified enzyme is introduced and expressed under suitable conditions using a carotenoid or apocarotenoid-producing host cell as specified herein, the formation of carotenoids could be increased by at least about 5-30%, the formation of apocarotenoids, particularly retinoids, could be increased by at least about 16-26%, such as e.g. 20, 25, 30% or more as compared to the corresponding process using the non-modified CarRP according to SEQ ID NO:1 or shown in FIG. 1.

In one embodiment, the modified CarRP enzyme as defined herein comprises an amino acid substitution at a position corresponding to residue 431 in the polypeptide according to SEQ ID NO:1 or shown in FIG. 1, particularly introduction of threonine, e.g. via substitution of serine (S431T), wherein the modified enzyme is derived from an enzyme with at least about 20% identity to McCarRP according to SEQ ID NO:1 or shown in FIG. 1. Using such modified enzyme comprising said mutation in a fermentation process, wherein said modified enzyme is introduced and expressed under suitable conditions using a carotenoid or apocarotenoid-producing host cell as specified herein, the formation of carotenoids could be increased by at least about 5-30%, the formation of apocarotenoids, particularly retinoids, could be increased by at least about 20-42%, such as e.g. 25, 30, 35, 40, 45% or more, as compared to the corresponding process using the non-modified CarRP according to SEQ ID NO:1 or shown in FIG. 1.

In one embodiment, the modified CarRP enzyme as defined herein comprises an amino acid substitution at a position corresponding to residue 432 in the polypeptide according to SEQ ID NO:1 or shown in FIG. 1, particularly introduction of isoleucine, e.g. via substitution of valine (V432I), wherein the modified enzyme is derived from an enzyme with at least about 20% identity to McCarRP according to SEQ ID NO:1 or shown in FIG. 1. Using such modified enzyme comprising said mutation in a fermentation process, wherein said modified enzyme is introduced and expressed under suitable conditions using a carotenoid or apocarotenoid-producing host cell as specified herein, the formation of carotenoids could be increased by at least about 5-30%, the formation of apocarotenoids, particularly retinoids, could be increased by at least about 20-65%, such as e.g. 25, 30, 35, 40, 45, 50, 55, 60, 65, 70% or more, as compared to the corresponding process using the non-modified CarRP according to SEQ ID NO:1 or shown in FIG. 1.

In one embodiment, the modified CarRP enzyme as defined herein comprises an amino acid substitution at a position corresponding to residue 476 in the polypeptide according to SEQ ID NO:1 or shown in FIG. 1, particularly introduction of alanine, e.g. via substitution of isoleucine (I476A), wherein the modified enzyme is derived from an enzyme with at least about 20% identity to McCarRP according to SEQ ID NO:1 or shown in FIG. 1. Using such modified enzyme comprising said mutation in a fermentation process, wherein said modified enzyme is introduced and expressed under suitable conditions using a carotenoid or apocarotenoid-producing host cell as specified herein, the formation of carotenoids could be increased by at least about 5-30%, the formation of apocarotenoids, particularly retinoids, could be increased by at least about 20-25%, as compared to the corresponding process using the non-modified CarRP according to SEQ ID NO:1 or shown in FIG. 1.

In one embodiment, the modified CarRP enzyme as defined herein comprises an amino acid substitution at a position corresponding to residue 547 in the polypeptide according to SEQ ID NO:1 or shown in FIG. 1, particularly introduction of isoleucine, e.g. via substitution of valine (V547I), wherein the modified enzyme is derived from an enzyme with at least about 20% identity to McCarRP according to SEQ ID NO:1 or shown in FIG. 1. Using such modified enzyme comprising said mutation in a fermentation process, wherein said modified enzyme is introduced and expressed under suitable conditions using a carotenoid or apocarotenoid-producing host cell as specified herein, the formation of carotenoids could be increased by at least about 5-30%, the formation of apocarotenoids, particularly retinoids, could be increased by at least about 20-35%, such as e.g. 25, 30, 35, 40% or more as compared to the corresponding process using the non-modified CarRP according to SEQ ID NO:1 or shown in FIG. 1.

In one embodiment, the modified CarRP enzyme as defined herein comprises an amino acid substitution at a position corresponding to residue 579 in the polypeptide according to SEQ ID NO:1 or shown in FIG. 1, particularly introduction of lysine, e.g. via substitution of arginine (R579K), wherein the modified enzyme is derived from an enzyme with at least about 20% identity to McCarRP according to SEQ ID NO:1 or shown in FIG. 1. Using such modified enzyme comprising said mutation in a fermentation process, wherein said modified enzyme is introduced and expressed under suitable conditions using a carotenoid or apocarotenoid-producing host cell as specified herein, the formation of carotenoids could be increased by at least about 5-30%, the formation of apocarotenoids, particularly retinoids, could be increased by at least about 20-38%, such as e.g. 25, 30, 35, 40% or more, as compared to the corresponding process using the non-modified CarRP according to SEQ ID NO:1 or shown in FIG. 1.

Thus, in some embodiment, the modified enzyme comprises two or more mutations, i.e. amino acid substitutions, such as e.g. at least 2, 3, 4, 5, 6, 7, 8, 9, 10, 11, 12, 13, 14, said mutations being selected from at least 2 to 14 mutations on positions corresponding to 7, 33, 153, 159, 167, 194, 305, 330, 430, 431, 432, 476, 547 or 579 in the polypeptide according to SEQ ID NO:1 or shown in FIG. 1 and wherein said at least 2-14 amino acids introduced on positions corresponding to said loci in the polypeptide according to SEQ ID NO:1 or shown in FIG. 1 are different from E7, Q7, A33, W33, A153, L159, Y167, 1194, T305, D330, S430, S431, V432, 1476, V547, R579 in any combination.

In one preferred embodiment, the present invention is directed to a modified CarRP derived from McCarRP as shown in SEQ ID NO:1 or shown in FIG. 1 and a method for producing said modified CarRP as described herein, said modified CarRP comprising 1 or 14 amino acid substitution(s) selected from the group consisting of E7D, A33N, A135S, L159V, Y167F, I194L, T305A, D330E or D330N, S430A, S431T, V432I, I476A, V547I, R579K, and combinations thereof. Expressing such modified enzyme in a suitable retinoid-producing host cell, the percentage of retinoids could be increased by 5 to 75%, such as e.g. 10, 15, 20, 25, 30, 35, 40, 45, 50, 55, 60, 65, 70, 75, 80% or more, and more compared to a process wherein the retinoid-producing host cell expresses a CarRP according to SEQ ID NO:1 or shown in FIG. 1. As used herein, the term “at least two mutations” means that the modified enzyme described herein comprises 2 or more mutations, such as e.g. 2, 3, 4, 5, 6, 7, 8, 9, 10, 11, 12, 13 or 14 mutations on locations corresponding to the specified position in the polypeptide according to SEQ ID NO:1 or shown in FIG. 1. Particularly, said modified CarRP enzmyes are expressed in an apocarotenoid, such as retinoid, and/or carotenoid producing host cell, such as preferably selected from Yarrowia, Saccharomyces or Escherichia, most preferably expressed in a carotenoid and/or apocarotenoid, such as retinoid, producing Yarrowia lipolytica as known in the art. Most preferably, said modified CarRPs as defined herein are codon-optimized for expression in the respective host cell.

Particularly, the present invention is directed to a modified CarRP derived from McCarRP as shown in SEQ ID NO:1 or shown in FIG. 1 including a protein with at least about 20% identity and a method for producing said modified CarRP as described herein, said modified CarRP comprising at least 1, 2, 3, 4, 5 amino acid substitution(s), preferably 5 amino acid substitutions selected from the group consisting of E7D, A33N, A135S, L159V, Y167F, I194L, T305A, D330E or D330N, S430A, S431T, V432I, I476A, V547I, and R579K, in any combination, more particularly wherein comprising combination of E7D with A33N, Y167F, V547I and R579K, or combination of E7D with A33N, Y167F, D330N, and V432I, or combination of E7D with D330N, S430A, V547I, and R579K, or combination of E7D with A33N, Y167F, T305A, and R576K, or combination of E7D with A33N, Y167F, S431T, and R579K, or combination of E7D, A33N, Y167F, S430A, and R579K, or combination of E7D with A33N, Y167F, S430A, and V547I, or combination of E7D with A33N, Y167F, T305A, and S430A, or combination of E7D with A33N, S430A, V547I, and R579K, or combination of E7D with A33N, Y167F, T305A, and D33N, or combination of A33N with Y167F, S430A, V547I, and R579K, or combination of E7D with Y167F, S430A, V547I, and R579K, or combination of E7D with Y167F, S431T, V547I, and R579K, or combination of E7D with A33N, Y167F, V431I and V547I.

In some preferred embodiments, the present invention is directed to a modified CarRP derived from McCarRP as shown in SEQ ID NO:1 or shown in FIG. 1 including a protein with at least about 20% identity and a method for producing said modified CarRP as described herein, said modified CarRP comprising at least one mutation, such as V432I, or combination of at least 5 mutations, comprising E7D in combination with A33N or Y167F, S430I, V547I and R579K, wherein through introduction of said amino acid substitutions and expression of said modified enzymes in a suitable apocarotenoid, such as retinoid, and/or carotenoid producing host cell, the percentage of e.g. retinoids can be increased by about 60-70% and more compared to a host cell expressing a non-modified CarRP as defined herein, such as e.g. McCarRP according to SEQ ID NO:1 or shown in FIG. 1.

According to all embodiments of the present invention, a modification in an amino acid corresponding to the specified position in the polypeptide according to SEQ ID NO:1 or shown in FIG. 1, such as e.g. an amino acid substitution on a position corresponding to residue 7, 33, 153, 159, 167, 194, 305, 330, 430, 431, 432, 476, 547 or 579 in the polypeptide according to SEQ ID NO:1 or shown in FIG. 1, wherein either one or more of said amino acids are modified, such as particularly at least 1, 2, 3, 4, 5 amino acids are modified, results in increased formation of carotenoids and/or apocarotenoids, preferably retinoids, when used in the respective host cell, i.e. a carotenoid and/or apocarotenoid-producing host cell as defined herein, wherein the titer of carotenoids or retinoids can be increased by at least about 5% as compared to the respective host cell wherein the specified amino acids are not substituted in a way as defined herein, i.e. compared to a host cell expressing a polypeptide according to SEQ ID NO:1 or shown in FIG. 1. Particularly, the titer of apocarotenoids, particularly retinoids, might be increased by about 5, 10, 15, 20, 25, 30, 35, 40, 45, 50, 55, 60, 65, 70, 75% or more, wherein an increase in retinoids of at least about 40 to 70% based on total retinoids is achieved with single mutations on positions corresponding to V432I, A33N, L159V, A153S in the polypeptide according to SEQ ID NO:1 or shown in FIG. 1, wherein an increase of at least about 25 to 75%, such as about 40 to 75% and more might be achieved using modified CarRP comprising quintuple mutations as defined herein, such as comprising one or more of amino acid substitutions corresponding to E7D, A33N, S430A, V547I, R579K, Y167F in the polypeptide according to SEQ ID NO:1 or shown in FIG. 1, and as compared to the corresponding host cell expressing a polypeptide according to SEQ ID NO:1 or shown in FIG. 1. Particularly, the titer of specific carotenoids, such as e.g. zeaxanthin, canthaxanthin, astaxanthin, rhodoxanthin, beta-cryptoxanthin might be increased by about 5, 10, 15, 20, 25, 30, 35, 40% or more, wherein an increase in total carotenoids of at least about 5 to 35% is achieved with single mutations on positions corresponding to V432I, V547I, S431T, D330E, T305 in the polypeptide according to SEQ ID NO:1 or shown in FIG. 1, wherein an increase in canthaxanthin of at least about 5 to 40% based on total carotenoids is achieved with single mutations on positions corresponding to V432I, V547I, S431T, D330E, T305 in the polypeptide according to SEQ ID NO:1 or shown in FIG. 1, and as compared to the corresponding host cell expressing a polypeptide according to SEQ ID NO:1 or shown in FIG. 1.

A modified host cell as defined herein comprises one or more copies of modified enzymes as defined herein, preferably wherein the modified enzymes are heterologous expressed in said modified host cell. Modifications in order to have the host cell as defined herein produce more copies of genes and/or proteins, such as e.g. more copies of modified CarRP may include the use of strong promoters, suitable transcriptional- and/or translational enhancers, or the introduction of one or more gene copies into the carotenoid and/or apocarotenoid-producing host cell, particularly fungal host cell, leading to increased accumulation of the respective enzymes in a given time. The skilled person knows which techniques to use depending on the host cell. The increase or reduction of gene expression can be measured by various methods, such as e.g. Northern, Southern or Western blot technology as known in the art.

The generation of a mutation into nucleic acids or amino acids, i.e. mutagenesis, may be performed in different ways, such as for instance by random or side-directed mutagenesis, physical damage caused by agents such as for instance radiation, chemical treatment, or insertion of a genetic element. The skilled person knows how to introduce mutations.

Thus, the present invention is directed to a carotenoid and/or apocarotenoid-producing host cell and a process for producing said host cells, particularly fungal host cell, as described herein comprising an expression vector or a polynucleotide encoding modified CarRP as described herein (see also WO2009126890, particularly Ex.1A) which has been integrated in the chromosomal DNA of said host cell. Such modified host cell, particularly fungal host cell, comprising a heterologous polynucleotide either on an expression vector or integrated into the chromosomal DNA encoding modified CarRP, as described herein is called a recombinant or modified host cell. The carotenoid and/or apocarotenoid-producing host cell, particularly fungal host cell, might contain one or more copies of a gene encoding the modified CarRP as defined herein, comprising the mutations as defined herein, leading to overexpression of such genes encoding said modified CarRP as defined herein. The increase of gene expression can be measured by various methods, such as e.g. Northern, Southern or Western blot technology, transcriptomics, genome sequencing or proteomics as known in the art.

The present invention is particularly directed to the use of such novel modified CarRPs in a process for production of carotenoids and/or apocarotenoids, particularly retinoids, including processes wherein the retinoids comprising a mix of retinol, retinal, retinyl acetate particularly with a percentage of at least about 40 wt % being retinyl acetate based on total retinoids and wherein particularly the formation of long-chain retinyl esters is reduced. The skilled person knows how to generate such conditions (see, e.g. WO2021136689 or WO2022090548).

The terms “sequence identity”, “% identity” are used interchangeable herein. For the purpose of this invention, it is defined here that in order to determine the percentage of sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/bases or amino acids. The sequence identity is the percentage of identical matches between the two sequences over the reported aligned region. The percent sequence identity between two amino acid sequences or between two nucleotide sequences may be determined using the Needleman and Wunsch algorithm for the alignment of two sequences (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, Longden and Bleasby, Trends in Genetics 16, (6) pp 276-277, http://emboss.bioinformatics.nl/). For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: number of corresponding positions in the alignment showing an identical amino acid or identical nucleotide in both sequences divided by the total length of the alignment after subtraction of the total number of gaps in the alignment. The identity as defined herein can be obtained from NEEDLE by using the NOBRIEF option and is labeled in the output of the program as “longest identity”. If both amino acid sequences which are compared do not differ in any of their amino acids, they are identical or have 100% identity.

The modified enzymes as defined herein can comprise further amino acid substitution(s) which do not alter enzyme activity, i.e. which show the same properties as phytoene synthase and/or lycopene beta cyclase with respect to the enzymes defined herein and catalyze the conversion of GGPP into phytoene and/or the conversion of lycopene into beta-carotene in the same range as the modified enzymes carrying only one or more of the amino acid substitution(s) as described herein. Such mutations are also called “silent mutations”, which do not alter the (enzymatic) activity of the enzymes according to the present invention.

Expression of the enzymes/polynucleotides encoding one of the modified CarRPs as defined herein can be done in any host system, including (micro) organisms, which is suitable for carotenoid and/or apocarotenoid production and which allows expression of the nucleic acids encoding one of the modified enzymes as disclosed herein, including functional equivalents or derivatives as described herein. Examples of suitable carotenoids/apocarotenoid-producing host (micro) organisms are bacteria, algae, fungi, including yeasts, plant or animal cells. Preferred bacteria are those of the genera Escherichia, such as, for example, Escherichia coli, Streptomyces, Pantoea (Erwinia), Bacillus, Flavobacterium, Synechococcus, Lactobacillus, Corynebacterium, Micrococcus, Mixococcus, Brevibacterium, Bradyrhizobium, Gordonia, Dietzia, Muricauda, Sphingomonas, Synochocystis, Paracoccus, such as, for example, Paracoccus zeaxanthinifaciens. Preferred eukaryotic microorganisms, in particular fungi including yeast, are selected from Saccharomyces, such as Saccharomyces cerevisiae, Aspergillus, such as Aspergillus niger, Pichia, such as Pichia pastoris, Hansenula, such as Hansenula polymorpha, Kluyveromyces, such as Kluyveromyces lactis, Phycomyces, such as Phycomyces blakesleanus, Mucor, Rhodotorula, Sporobolomyces, Xanthophyllomyces, Phaffia, Blakeslea, such as e.g. Blakeslea trispora, or Yarrowia, such as Yarrowia lipolytica. In particularly preferred is expression in a fungal host cell, such as e.g. Yarrowia or Saccharomyces, or expression in Escherichia, more preferably expression in Yarrowia lipolytica or Saccharomyces cerevisiae.

With regards to the present invention, it is understood that organisms, such as e.g. microorganisms, fungi, algae or plants also include synonyms or basonyms of such species having the same physiological properties, as defined by the International Code of Nomenclature of Prokaryotes or the International Code of Nomenclature for algae, fungi, and plants (Melbourne Code). Thus, for example, strain Lachancea mirantina is a synonym of strain Zygosaccharomyces sp. IFO 11066, originated from Japan.

In some embodiments, the present invention is directed to production of retinoids, including but not limited to production of retinal, retinol, retinyl acetate as described in e.g. WO2019058001, with a percentage of retinyl acetate of at least about 40-80 wt % based on total retinoids, using a host cell expressing a modified CarRP enzyme as described herein and wherein the production of retinoids is increased by at least about 5%, such as 10, 15, 20, 25, 30, 35, 40, 45, 50, 55, 60, 65, 70, 75% and more as compared to a process using the corresponding non-modified CarRP enzyme according to SEQ ID NO:1 or shown in FIG. 1. The produced retinyl acetate might be isolated and optionally further purified from the medium and/or host cell. Said acetylated retinoids defined herein can be used as building blocks leading to vitamin A.

Modified host cells as defined herein may be cultured in an aqueous medium supplemented with appropriate nutrients under aerobic or anaerobic conditions and as known by the skilled person for the respective carotenoids and/or apocarotenoids-producing host cells. Optionally, such cultivation is in the presence of proteins and/or co-factors involved in transfer of electrons, as known in the art. Suitable carbon sources for the purpose of the present invention might be selected from glucose, fructose, raffinose, lactose, galactose, glycerol, xylose, arabinose, sucrose, maltose, vegetable oil including but not limited to oleic or linoleic acid, with or without the presence of ethanol, particularly selected from glucose, galactose or xylose. Particular culture conditions for production of apocarotenoids, particularly retinoids, might contain a batch and feed run, with a concentration of 5% (w/v) glucose and 1% ethanol (w/v) in the batch phase and a concentration of 100% (w/v) in the feeding phase. The cultivation/growth of the host cell may be conducted in batch, fed-batch, semi-continuous or continuous mode, particularly in fed-batch mode for 80, 90, 100, 110, 120, 130 h under suitable culture conditions. Depending on the host cell and the carotenoid and/or apocarotenoid to be produced conditions might be adapted as it is known to the skilled person.

Cultivation and isolation of carotenoid and/or apocarotenoid-producing host cells, such as e.g. selected from Yarrowia and Saccharomyces is described in e.g. WO2008042338. With regards to production of beta-carotene and retinoids in host cells selected from E. coli, methods are described in e.g. US20070166782.

In some embodiments, apocarotenoid-producing host cells expressing the modified CarRPs as defined herein are cultivated in a two-phase system, wherein the apocarotenoids, particularly retinoids, including but not limited to retinol and/or retinyl acetate, preferably with at least about 40-80 wt % retinyl acetate based on total retinoids, are collected in and afterwards isolated from a suitable lipophilic phase. Particular conditions and lipophilic solvents are disclosed in WO2022090548 or WO2022090549.

In some embodiments, the present invention is directed to a two-phase fermentation with retinoid-producing strains expressing modified CarRPs as defined herein using lipophilic solvents as second phase, such as e.g. isopars or corn oil, besides the known solvents comprising Drakeol®, silicone or n-dodecane (see Jang et al., Microbial Cell Factories 10:59, 2011).

As used herein, the term “specific activity” or “activity” with regards to enzymes means its catalytic activity, i.e. its ability to catalyze formation of a product from a given substrate. The specific activity defines the amount of substrate consumed and/or product produced in a given time period and per defined amount of protein at a defined temperature. Typically, specific activity is expressed in μmol substrate consumed or product formed per min per mg of protein. Typically, μmol/min is abbreviated by U (=unit). Therefore, the unit definitions for specific activity of μmol/min/(mg of protein) or U/(mg of protein) are used interchangeably throughout this document. An enzyme is active, if it performs its catalytic activity in vivo, i.e. within the host cell as defined herein or within a suitable (cell-free) system in the presence of a suitable substrate. The skilled person knows how to measure enzyme activity. Analytical methods to evaluate the capability of a suitable bi-functional CarRPs as defined herein are known in the art, such as e.g. described in Ma et al. (Nature Communications, 2022, 13:572, https://doi.org/10.1038/s41467-022-28277-w). Titers of products including apocarotenoids or carotenoids, such as retinyl acetate, retinol, trans-retinal, cis-retinal, beta-carotene, canthaxanthin, zeaxanthin, astaxanthin, rhodoxanthin, lycopene, phytoene, beta-ionone and the like can be measured by HPLC.

The general construction of carotenoid-producing host cells, particularly beta-carotene producing host cells is known in the art, such as e.g. described in WO2006102342.

As used herein, a “retinoid-producing host cell” is a specific apocarotenoid-producing host cell, wherein the respective polypeptides are expressed and active in vivo, leading to production of retinoids as defined herein, e.g. including retinal, retinol and/or retinyl acetate, via enzymatic conversion of beta-carotene via retinal into retinol and optionally further into retinyl acetate. These polypeptides include enzymes catalyzing the conversion of beta-carotene into retinal, see e.g. WO2019057999, catalyzing the conversion of retinal into retinol, see e.g. WO2019057998, and optionally catalyzing the conversion of retinol into retinyl acetate, see e.g. WO2019058001.

“Vitamin A” as used herein may be any chemical form of vitamin A found in aqueous solutions, in solids and formulations, and includes retinol, retinyl acetate and retinyl esters. It also includes retinoic acid, such as for instance undissociated, in its free acid form or dissociated as an anion.

“Retinal” as used herein is known under IUPAC name (2E,4E,6E,8E)-3,7-Dimethyl-9-(2,6,6-trimethylcyclohexen-1-yl)-nona-2,4,6,8-tetraenal and includes both cis- and trans-isoforms, such as e.g. 11-cis retinal, 13-cis retinal, trans-retinal and all-trans retinal.

### FIGURES

FIG. 1: Amino acid sequence of M. circinelloides CarRP (McCarRP; SEQ ID NO:1) wherein the amino acid residues selected for amino acid substitutions as described in the present application are marked in bold/underlined and wherein the amino acids replacing the original amino acids as shown in SEQ ID NO:1 are marked in italic and shown on top of the respective original amino acids.

The following examples are illustrative only and are not intended to limit the scope of the invention in any way. The contents of all references, patent applications, patents, and published patent applications, cited throughout this application are hereby incorporated by reference, in particular, WO2014096992, WO2019058001, WO2021136689, WO2022090548, WO2008042338, US20070166782, WO2022090549, WO2006102342, WO2016172282, WO2019058000, WO2019057999, WO2019057998, and US20180148697.

### EXAMPLES

### Example 1: General Methods, Strains, and Plasmids

All basic molecular biology and DNA manipulation procedures described herein are generally performed according to Sambrook et al. (eds.), Molecular Cloning: A Laboratory Manual. Cold Spring Harbor Laboratory Press: New York (1989) or Ausubel et al. (eds). Current Protocols in Molecular Biology. Wiley: New York (1998).

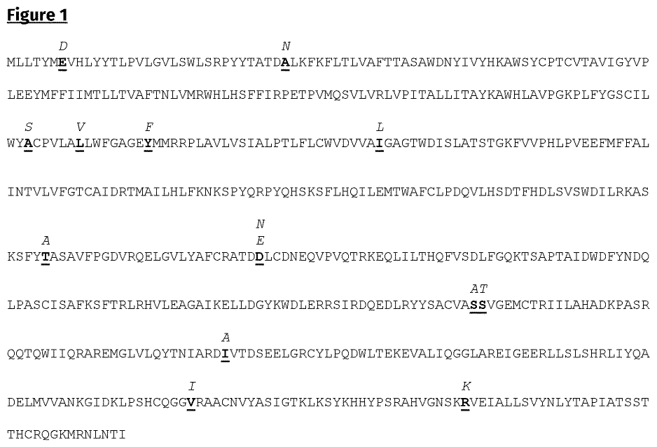

Strains, plasmids, and sequences. Strains used as host cells and the respective plasmids used for expression of CarRP and other enzymes or constructs specified below are listed in Table 1, 2 and 4 and in the sequence listing. FIG. 1 shows the wild-type CarRP (McCarRP) amino acid sequence (SEQ ID NO:1) with specific indication of the residues selected for modification as defined herein.

TABLE 1
list of Yarrowia strains (indicated with “ML”) used for production
of carotenoids and/or retinoids carrying the heterologous (non-
modified or modified) CarRP genes. For more details, see text.

| Strain | Description | First described in |
|---|---|---|
| ML18743 | ML18667 transformed with MB9523 | WO2022090548 |
| ML19637 | | this application |
| ML19836 | | this application |
| ML19836-ura | | this application |
| ML15710 | | WO2016172282 |

TABLE 2
list of plasmids used for construction of the strains carrying
heterologous genes expressing the inserts as specified (“Insert”) including
polynucleotide sequence expressing McCarRP according to
SEQ ID NO: 1 (SEQ ID NO: 2) and other genes used for retinoid
and/or carotenoid production. All modified CarRPs are based
on McCarRP according to SEQ ID NO: 1 or shown in FIG. 1.
For more details, see text.

| Plasmid | Insert | Marker |
|---|---|---|
| MB10157 | MccarRP | URA3 + ARG4 |
| MB10866 | Cas9 + sgCarRP | HygR |
| MB9282 | Cas9 + sgKu70 | HygR |
| MB6128 | CRE-recombinase | G418/Geneticin |
| MB7076 | crtW | HygR |
| MB7190 | crtZ | NatR |
| MB7082 | crtW | NatR |
| MB9930 | crtZ | HygR |
| MB7522 | Cas9 + sgcarB | HygR |
| MB9931 | Lfreq-crtZ | HygR |
| MB7918 | bhy-21 | HygR |
| MB6806 | cCD1 | HygR |

UPLC reverse phase method. For rapid screening this method does not separate cis-isomers, only major functional groups. A Waters Acquity UPLC with PDA detection (or similar) with auto sampler was used to inject samples. An Acquity UPLC HSS T3 1.8 μm P/N 186003539 was used to resolve retinoids and/or carotenoids. The mobile phase consisted of either, 1000 mL hexane, 30 mL isopropanol, and 0.1 mL acetic acid for retinoid related compounds including carotenoids. The flow rate for each was 0.6 mL per minute. Column temperature was 20° C. The injection volume was 5 μL. The detector was a photodiode array detector collecting from 210 to 600 nm. Analytes were detected according to Table 3. Rhodoxanthin, astaxanthin, zeaxanthin and ionones can be quantified as in the following references respectively: Royer et al., (Sci. Adv. 2020; Vol. 6, Issue 17), WO2014096992, and US20180148697.

TABLE 3A
list of analytes using reverse phase method. The addition of
all added intermediates gives the total amount retinoids or
carotenoids depending on the measurement. Beta-carotene* can
be detected in 325 nm and will interfere with retinyl ester
quantitation, therefore care must be taken to observe the carotene
peak and not include them in the retinoid quantification. “N/A”
means “not available”. For more details, see text.

| | Retention | | |
|---|---|---|---|
| | time | Lambda max | Response |
| Intermediates | [min] | [nm] | factor |
| retinyl-acetate | 2.93 | 325 | 1.00 |
| retinyl-esters | 3.2-3.8 | 325 | 1.68 |
| retinal | 2.77 | 325 | 0.87 |
| retinol | 2.73 | 325 | 0.87 |
| Beta-carotene* | 3.56 | 450 | N/A |
| Canthaxanthin | 3.12 | 470 | N/A |

TABLE 3B
UPLC Method Gradient with solvent A: water;
solvent B: acetonitrile; solvent C: methanol;
solvent D: tert-butyl methyl ether.

| Time | % | % | % | % | Flow | Pressure |
|---|---|---|---|---|---|---|
| [min] | A | B | C | D | [ml/min] | [psi/bar] |
| 0 | 50 | 50 | 0 | 0 | 0.5 | 9500-14000 max |
| 0.5 | 50 | 50 | 0 | 0 | 0.5 | |
| 1.0 | 0 | 50 | 50 | 0 | 0.5 | |
| 1.25 | 0 | 0 | 100 | 0 | 0.5 | |
| 3.25 | 0 | 0 | 5 | 95 | 0.5 | |
| 3.5 | 0 | 0 | 5 | 95 | 0.5 | |
| 4.0 | 0 | 0 | 100 | 0 | 0.5 | |
| 4.25 | 0 | 50 | 50 | 0 | 0.5 | |
| 4.5 | 50 | 50 | 0 | 0 | 0.5 | |

Method Calibration. Method is calibrated on carotenoids, retinyl acetate, retinols and retinals are quantitated against retinyl-acetate using the indicated response factor. Retinyl Acetate is dissolved in THF at ~200 μg/ml for stock solution using a volumetric flask. Using volumetric flasks, ×20, ×50 and ×100 dilutions of stock solution in 50/50 methanol/MTBE were made. UV absorbance of retinyl acetate becomes nonlinear fairly quickly, so care must be taken to stay within the linear range. Consequently, lower concentrations might be better. Retinyl palmitate can also be used as retinyl ester calibration. Peaks for retinyl acetate at about 3 minutes and peaks for retinyl esters (long-chain retinyl esters) at around 3.5 minutes. For measurement of other apocarotenoids or carotenoids, this can be adapted accordingly.

Sample preparation. Samples were prepared by various methods depending on the conditions. For whole broth or washed broth samples the broth was placed in a Precellys® tube, weighed, and mobile phase was added. Briefly in a 2 ml Precellys® tube, add 25 μl of well mixed broth and 975 μl of THF. The samples were then processed in a Precellys® homogenizer (Bertin Corp, Rockville, MD, USA) on the highest setting 3× according to the manufacturer's directions, typically 3×15×7500 tpms. For the washed pellet the samples were spun in a 1.7 ml tube in a microfuge at 10000 rpm for 1 minute, the broth decanted, 1 ml water added, mixed, pelleted and decanted, and brought up to the original volume. The mixture was pelleted again and brought up in appropriate amount of mobile phase and processed by Precellys® bead beating. For analysis of silicone oil fraction, the sample was spun at 4000 RPM for 10 minutes and the oil was decanted off the top by positive displacement pipet (Eppendorf, Hauppauge, NY, USA) and diluted into mobile phase mixed by vortexing and measured for retinoid (or other compounds to be measured) concentration by UPLC analysis.

### Example 2: Production of Retinoids in Yarrowia lipolytica Expressing Mutant carRP

For evaluation of carRP alleles, strain ML18743 was transformed with plasmid MB10866 (SEQ ID NO:4), which codes for a synthetic guide RNA (sgRNA) and SpCas9 protein to direct mutagenesis at carRP sequences present in the genome. Strain ML19637, mutant for carRP, was identified by virtue of its white color. Strain ML19637 was passaged onto non-selective media, and hygromycin-sensitive isolates were identified. One such isolate was further mutagenized by plasmid MB9282 (SEQ ID NO:5), which encodes for a sgRNA and SpCas9 protein to direct mutagenesis at the Ku70 locus, to create ML19836. Uracil auxotroph strain ML19836-ura was isolated from ML19836 by selection on 5-fluoroorotic acid (5-FOA) containing media. Strain ML19836-ura was transformed with Sfil-linearized DNA from plasmid MB10157 (SEQ ID NO:3) expressing the wild-type McCarRP and mutant derivatives MB10157-1 to MB10157-29 shown in Table 4A and 4B and as highlighted in bold/underlined in the polypeptide sequence shown in FIG. 1 and selected for uracil prototrophy.

Transformants were grown in shake plates as described elsewhere, see e.g. WO2022090549. Typically, 200 μl of 0.075% Yeast extract, 0.25% peptone (0.25×YP) is inoculated with 10 μl of freshly grown Yarrowia and overlaid with 200 μl of Drakeol 5 (Penreco, Karns City, PA, USA) mineral oil, silicone oil, or corn oil with either 2% oleic acid or 2% glucose as a carbon source. Clonal isolates of transformants were grown in 24 well plates (Multitron, 30° C., 800 RPM) in YPD media with one of the overlays indicated earlier for 4 days. The overlay fraction was removed from the shake plate wells and analyzed by HPLC on a normal phase column, with a photo-diode array detector. Retinoid production was measured (Table 4), wherein the percentage of retinoids (titer of total retinoids from shake plate assay described above) using the polynucleotide expressing reference MccarRP according to SEQ ID NO:1 or FIG. 1 expressed on plasmid MB10157 is set to 100%.

TABLE 4A
Impact of carRP alleles on retinoid output as a fraction of the output
or titer obtained using the wildtype carRP according to SEQ ID NO:
2 (plasmid MB10157 expressing wt CarRP according to SEQ ID NO: 1
or FIG. 1) compared to single mutations of CarRP (e.g. E7D, A33N,
etc.; plasmids MB10157-1 to MB10157-15 carrying the mutated carRP
gene) as indicated as “insert”. For more details, see text.

| Plasmid | Insert | Retinoids [%] |
|---|---|---|
| MB10157 | MccarRP | 100 |
| MB10157-1 | MccarRP_E7D | 105 |
| MB10157-2 | MccarRP_A33N | 146 |
| MB10157-3 | MccarRP_A153S | 143 |
| MB10157-4 | MccarRP_L159V | 141 |
| MB10157-5 | MccarRP_Y167F | 124 |
| MB10157-6 | MccarRP_I194L | 135 |
| MB10157-7 | MccarRP_T305A | 123 |
| MB10157-8 | MccarRP_D330E | 136 |
| MB10157-9 | MccarRP_D330N | 105 |
| MB10157-10 | MccarRP_S430A | 116 |
| MB10157-11 | MccarRP_S431T | 130 |
| MB10157-12 | MccarRP_V432I | 165 |
| MB10157-13 | MccarRP_I476A | 125 |
| MB10157-14 | MccarRP_V547I | 135 |
| MB10157-15 | MccarRP_R579K | 138 |

TABLE 4B
Impact of carRP alleles on retinoid output as a fraction of the output of titer
obtained using the wildtype carRP according to SEQ ID NO: 2 (plasmid MB10157
expressing wt CarRP according to SEQ ID NO: 1 or FIG. 1) compared to quintuple
mutations of carRP (plasmids MB10157-16 to MB10157-29 carrying quintuple mutated
carRP gene) as indicated as “insert”. For more details, see text.

| | | Retinoids |
|---|---|---|
| Plasmid | Insert | [%] |
| MB10157 | MccarRP | 100 |
| MB10157-16 | MccarRP_E7D_A33N_Y167F_V547I_R579K | 159 |
| MB10157-17 | MccarRP_E7D_A33N_Y167F_D330N_V432I | 149 |
| MB10157-18 | MccarRP_E7D_D330N_S430A_V547I_R579K | 147 |
| MB10157-19 | MccarRP_E7D_A33N_Y167F_T305A_R579K | 125 |
| MB10157-20 | MccarRP_E7D_A33N_Y167F_S431T_R579K | 148 |
| MB10157-21 | MccarRP_E7D_A33N_Y167F_S430A_R579K | 146 |
| MB10157-22 | MccarRP_E7D_A33N_Y167F_S430A_V547I | 138 |
| MB10157-23 | MccarRP_E7D_A33N_Y167F_T305A_S430A | 135 |
| MB10157-24 | MccarRP_E7D_A33N_S430A_V547I_R579K | 175 |
| MB10157-25 | MccarRP_E7D_A33N_Y167F_T305A_D330N | 128 |
| MB10157-26 | MccarRP_A33N_Y167F_S430A_V547I_R579K | 155 |
| MB10157-27 | MccarRP_E7D_Y167F_S430A_V547I_R579K | 172 |
| MB10157-28 | MccarRP_E7D_Y167F_S431T_V547I_R579K | 151 |
| MB10157-29 | MccarRP_E7D_A33N_Y167F_V432I_V547I | 136 |

### Example 3: Production of Various Carotenoids or Apocarotenoids in Yarrowia lipolytica Expressing Mutant carRP

Strains expressing mutant CarRP as well as specific genes for production of canthaxanthin, zeaxanthin, beta-cryptoxanthin, rhodoxanthin or beta-ionone are constructed as follows:

Beta-carotene-producing strain ML15710 is transformed with plasmid MB6128 (SEQ ID NO:6) containing the CRE-recombinase and is selected on geneticin-containing medium. Hygromycin-sensitive isolates are identified among the transformants by replica plating onto selective and nonselective medium. One hygromycin-sensitive isolate is further propagated on non-selective medium, from which geneticin-sensitive isolates are identified by replica plating onto selective and non-selective medium. One such isolate is transformed with one of the following Pvull-linearized plasmids: MB7076 (for production of canthaxanthin; SEQ ID NO:7), MB7190 (for production of zeaxanthin; SEQ ID NO:8), MB9931 (for production of beta-cryptoxanthin; SEQ ID NO:9), MB7918 (for production of rhodoxanthin; SEQ ID NO:10) or MB6806 (for production of beta-ionone; SEQ ID NO:11) and selected on hygromycin containing medium to create a canthaxanthin producing strain ML15710+crtW, zeaxanthin producing strain ML15710+crtZ, beta-cryptoxanthin producing strain ML15710+Lfreq-CrtZ, rhodoxanthin producing strain ML15710+bhy-21, or beta-ionone producing strain ML15710+CCD1. These strains are transformed with plasmid MB6128 containing the CRE-recombinase and selected on geneticin-containing medium. Hygromycin-sensitive isolates are identified among the transformants by replica plating onto selective and nonselective medium. One hygromycin-sensitive isolate (for every carotenoid/apocarotenoid as specified above) is further propagated on non-selective medium, from which geneticin-sensitive isolates are identified by replica plating onto selective and non-selective medium. These strains are then transformed with MB10866 as described in Example 1, to generate white carRP mutant strains ML15710+crtW+carRP⁻ (for canthaxanthin production), ML15710+crtZ+carRP⁻ (for zeaxanthin production), ML15710+Lfreq-CrtZ+carRP (for beta-cryptoxanthin production), ML15710+bhy-21+carRP⁻ (for rhodoxanthin production), or ML15710+CCD1+carRP⁻ (for beta-ionone production). Hygromycin-sensitive isolates are identified by passage on nonselective medium and replica plating to selective and non-selective medium. These strains are then transformed with plasmid MB9282 as in Example 1 to create ku70⁻ mutant strains ML15710+crtW+carRP+ku70⁻ (for canthaxanthin production), ML15710+crtZ+carRP+ku70⁻ (for zeaxanthin production), ML15710+Lfreq-CrtZ+carRP+ku70⁻ (for beta-cryptoxanthin production), ML15710+bhy-21+carRP+ku70⁻ (for rhodoxanthin production) or ML15710+CCD1+carRP+ku70⁻ (for beta-ionone production). Strains auxotrophic for uracil are isolated from these strains by selection on 5-FOA and are called ML15710+xx+carRP^{-+ku}70⁻+ura3⁻, wherein “xx” stands for the respective-carotenoid/apocarotenoid-specific gene(s). This white, uracil auxotrophic strain is then transformed with the plasmid MB10157 expressing the wild-type McCarRP as well as with the respective plasmids expressing mutated forms of McCarRP as listed in Table 4 (see Example 2), to generate canthaxanthin, zeaxanthin, beta-cryptoxanthin, rhodoxanthin or beta-ionone producing strains further expressing heterologous CarRPs (wt or mutated). Strains are cultivated in microtiter plates or fermentation as described in more detail below for canthaxanthin-producing strain but being mutatis mutandis applicable for production of other carotenoids such as zeaxanthin, beta-cryptoxanthin, rhodoxanthn or beta-ionone:

Transformants from strain ML15710+crtW+CarRP+ku70⁻+ura3⁻ with plasmid MB101570 or plasmids indicated in Table 5 were grown in microtiter plates as described in WO2022090549 or Example 2 above except for the use of a second phase and the use of glucose as sole carbon source. Fermentation and carotenoid analysis was performed according to the methods described previously (see e.g. U.S. Pat. No. 7,851,199 Ex.2-4). Introduction of plasmids MB101570-7, MB10157-11, and MB10157-14 resulted in an increase in the percentage of total carotenoids (titer of total carotenoids) as measured via shake plate assay described above and in the percentage of both total carotenoid yield and yield of canthaxanthin as measured via fermentation, i.e. yield on carbon (g total carotenoids/g carbon source and g canthaxanthin/g carbon source, respectively), wherein the percentage using the polynucleotide expressing reference MccarRP according to SEQ ID NO:1 or FIG. 1 expressed on plasmid MB10157 is set to 100%.

TABLE 5
Impact of CarRP alleles on carotenoid output as a fraction
of output of titer using the wildtype carRP according to SEQ
ID NO: 2 (plasmid MB10157 expressing wt CarRP according to
SEQ ID NO: 1 or FIG. 1) compared to single mutations of carRP
as indicated as “insert”. “CXN” means canthaxanthin.
“Carotenoids [%]” reflects measurement of carotenoid
titer (total carotenoids) from a microtiter plate. “Cartenoid
yield [%]” and “CXN yield [%]” means
the yield on carbon (g product/g carbon source) measured in
a fermentation. For more details, see text.

| | | Carotenoids | Carotenoid | CXN yield |
|---|---|---|---|---|
| Plasmid | Insert | [%] | yield [%] | [%] |
| MB10157 | MccarRP | 100 | 100 | 100 |
| MB10157-7 | MccarRP_T305A | 106 | 123 | 129 |
| MB10157-8 | MccarRP_D330E | n.a. | 118 | 120 |
| MB10157-11 | MccarRP_S431T | 108 | 100 | 100 |
| MB10157-12 | MccarRP_V432I | n.a. | 105 | 106 |
| MB10157-14 | MccarRP_V547I | 111 | 135 | 137 |

### Example 4: Production of Astaxanthin in Yarrowia lipolytica Expressing Mutant carRP

Beta-carotene-producing strain ML15710 is transformed with plasmid MB6128 containing the CRE-recombinase and is selected on geneticin-containing medium. Hygromycin-sensitive isolates are identified among the transformants by replica plating onto selective and nonselective medium. One hygromycin-sensitive isolate is further propagated on non-selective medium, from which geneticin-sensitive isolates are identified by replica plating onto selective and non-selective medium. One such isolate is transformed with Pvull-linearized plasmid MB7082 (SEQ ID NO:12) and selected on nourseothricin containing medium to create an astaxanthin producing strain ML15710+crtW. This strain is then transformed with Pvull-linearized MB9930 (SEQ ID NO:13) and selected on hygromycin media to generate ML15710+crtW+crtZ. This strain is transformed with plasmid MB6128 containing the CRE-recombinase and is selected on geneticin-containing medium. Hygromycin- and nourseothricin-sensitive isolates are identified among the transformants by replica plating onto selective and nonselective medium. One hygromycin- and nourseothricin-sensitive isolate is further propagated on non-selective medium, from which geneticin-sensitive isolates are identified by replica plating onto selective and non-selective medium. This strain is then transformed with MB10866 as described in Example 1, to generate a white carRP mutant strain ML15710+crtW+crtZ+carRP⁻. A hygromycin-sensitive isolate is identified by passage on nonselective medium and replica plating to selective and non-selective medium. This strain is then transformed with MB9282 as in Example 1 to create a ku70⁻ mutant strain ML15710+crtW+crtZ+carRP⁻+ku70⁻. A strain auxotrophic for uracil is isolated from this strain by selection on 5-FOA and is called ML15710+crtW+crtZ+carRP⁻+ku70⁻+ura3⁻. This white, uracil auxotrophic strain is then transformed with the plasmids expressing heterologous wt CarRP or mutants as listed in Table 4 (Example 2), to generate astaxanthin producing strains further expressing the mutated CarRPs. Compared to astaxanthin producing strains expressing the wt McCarRP according to SEQ ID NO:1 or FIG. 1, the titer of astaxanthin can be increased by at least about 5% (not shown) in a strain expressing CarRP mutants.

### Example 5: Production of Lycopene in Yarrowia lipolytica Expressing Mutant carRP

CarRP mutant expressing DNAs described in Table 2 are combined with the mutation E78G by DNA-synthesis provider (Genscript). This E78G mutation inactivates the lycopene cyclase domain of CarRP (see e.g. WO2014151748). Beta-carotene-producing strain ML15710 is transformed with plasmid MB6128 containing the CRE-recombinase and is selected on geneticin-containing medium. Hygromycin-sensitive isolates are identified among the transformants by replica plating onto selective and nonselective medium. One hygromycin-sensitive isolate is further propagated on non-selective medium, from which geneticin-sensitive isolates were identified by replica plating onto selective and non-selective medium. One such isolate is transformed with MB10866 as described in Example 1, to generate a white carRP mutant strain ML15710+carRP⁻. A hygromycin-sensitive isolate is identified by passage on nonselective medium and replica plating to selective and non-selective medium. This strain is then transformed with MB9282 as in Example 1 to create a ku70 mutant strain ML15710+carRP⁻+ku70⁻. A strain auxotrophic for uracil is isolated from this strain by selection on 5-FOA and is called ML15710+carRP⁻+ku70⁻+ura3⁻. This strain is transformed with the Sfil-linearized DNAs containing the mutations in Table 2 combined with the E78G mutation to create lycopene producing strains further expressing the mutated CarRPs. This strain is then transformed with the plasmids expressing heterologous wt CarRP or mutants as listed in Table 4 (Example 2) combined with E78G mutation, to generate lycopen producing strains further expressing the mutated CarRPs. Compared to lycopene producing strains expressing the wt McCarRP according to SEQ ID NO:1 or FIG. 1, the titer of lycopene can be increased by at least about 5% (not shown) in a strain expressing CarRP mutants.

### Example 7: Production of Phytoene in Yarrowia lipolytica Expressing Mutant carRP

Beta-carotene-producing strain ML15710 is transformed with plasmid MB6128 containing the CRE-recombinase and is selected on geneticin-containing medium. Hygromycin-sensitive isolates are identified among the transformants by replica plating onto selective and nonselective medium. One hygromycin-sensitive isolate is further propagated on non-selective medium, from which geneticin-sensitive isolates are identified by replica plating onto selective and non-selective medium. One such isolate is then transformed with MB10866 as described in Example 1, to generate a white carRP mutant strain ML15710+carRP⁻. A hygromycin-sensitive isolate is identified by passage on nonselective medium and replica plating to selective and non-selective medium. One such isolate is then transformed with MB7522 (SEQ ID NO:14) to generate carB mutant strain ML15710+carRP⁻+carB⁻. A hygromycin-sensitive isolate is identified by passage on nonselective medium and replica plating to selective and non-selective medium. This strain is then transformed with MB9282 as in Example 1 to create a ku70 mutant strain ML15710+carRP⁻+carB⁻+ku70⁻. A strain auxotrophic for uracil is isolated from this strain by selection on 5-FOA, and is called ML15710+carRP⁻+carB⁻+ku70⁻+ura3⁻. This white, uracil auxotrophic strain is then transformed with the plasmids expressing heterologous wt CarRP or mutants as listed in Table 4 (Example 2), to generate phytoene producing strains further expressing the mutated CarRPs. Compared to phytoene producing strains expressing the wt McCarRP according to SEQ ID NO:1 or FIG. 1, the titer of phytoene can be increased by at least about 8% (not shown) in a strain expressing CarRP mutants.

### Example 8: Production of Beta-Carotene in Yarrowia lipolytica Expressing Mutant carRP

Beta-carotene-producing strain ML15710 is transformed with plasmid MB6128 containing the CRE-recombinase and is selected on geneticin-containing medium. Hygromycin-sensitive isolates are identified among the transformants by replica plating onto selective and nonselective medium. One hygromycin-sensitive isolate is further propagated on non-selective medium, from which geneticin-sensitive isolates are identified by replica plating onto selective and non-selective medium. One such isolate is then transformed with MB10866 as described in Example 1, to generate a white carRP mutant strain ML15710+carRP⁻. A hygromycin-sensitive isolate is identified by passage on nonselective medium and replica plating to selective and non-selective medium. One such isolate is then transformed with MB9282 as in Example 1 to create a ku70⁻ mutant strain ML15710+carRP⁻+ku70⁻. A strain auxotrophic for uracil is isolated from this strain by selection on 5-FOA, and is called ML15710+carRP⁻+ku70⁻+ura3⁻. This white, uracil auxotrophic strain is then transformed with the plasmids expressing heterologous wt CarRP or mutants as listed in Table 4 (Example 2), to generate beta-carotene producing strains further expressing the mutated CarRPs. Compared to beta-carotene producing strains expressing the wt McCarRP according to SEQ ID NO:1 or FIG. 1, the titer of beta-carotene can be increased by at least about 5% (not shown) in a strain expressing CarRP mutants.

1-14. (canceled)

15. A modified bi-functional enzyme catalyzing the conversion of geranylgeranyl pyrophosphate (GGPP) into phytoene and/or conversion of lycopene into beta-carotene, said enzyme comprising one or more amino acid substitution(s), such as amino acid substitution(s) being introduced into a sequence with at least about 20%, such as 25, 30, 35, 40, 45, 50, 55, 60, 65, 70, 75, 80, 85, 90, 92, 95, 97, 98, 99% or up to 100% identity to SEQ ID NO:1 or shown in FIG. 1, said one or more amino acid substitution(s) being introduced at position(s) corresponding to amino acid residue(s) selected from the group consisting of position 7, 33, 153, 159, 167, 194, 305, 330, 430, 431, 432, 476, 547, 579, and combinations thereof, in the polypeptide according to SEQ ID NO:1 or shown in FIG. 1, and wherein the catalytic activity towards production of apocarotenoids, particularly retinoids, or carotenoids is increased by at least about 5% when expressed in a suitable apocarotenoid/retinoid-producing host cell as compared to the corresponding host cell expressing CarRP according to SEQ ID NO:1 or shown in FIG. 1.

16. The modified enzyme according to claim 15, wherein the amino acid residue on position corresponding to position 7 in SEQ ID NO:1 or shown in FIG. 1 is different from glutamic acid or glutamine, wherein the amino acid residue on position corresponding to position 33, in SEQ ID NO:1 or shown in FIG. 1 is different from alanine or tryptophane, wherein the amino acid residue on position corresponding to position 153 in SEQ ID NO:1 or shown in FIG. 1 is different from alanine, wherein the amino acid residue on position corresponding to position 159 in SEQ ID NO:1 or shown in FIG. 1 is different from leucine, wherein the amino acid residue on position corresponding to position 167 in SEQ ID NO:1 or shown in FIG. 1 is different from tyrosine, wherein the amino acid residue on position corresponding to position 194 or 476 in SEQ ID NO:1 or shown in FIG. 1 is different from isoleucine, wherein the amino acid residue on position corresponding to position 305 in SEQ ID NO:1 or shown in FIG. 1 is different from threonine, wherein the amino acid residue on position corresponding to position 330 in SEQ ID NO:1 or shown in FIG. 1 is different from aspartic acid, wherein the amino acid residue on position corresponding to position 430 or 431 in SEQ ID NO:1 or shown in FIG. 1 is different from serine, wherein the amino acid residue on position corresponding to position 432 or 547 in SEQ ID NO:1 or shown in FIG. 1 is different from valine, and/or wherein the amino acid residue on position corresponding to position 579 in SEQ ID NO:1 or shown in FIG. 1 is different from arginine.

17. The modified enzyme according to claim 15, wherein the amino acid residues corresponding to position(s) 7, 33, 153, 159, 167, 194, 305, 330, 430, 431, 432, 476, 547, 579, and/or combinations thereof in the polypeptide according to SEQ ID NO:1 or shown in FIG. 1 are selected from D7, N33, S153, V159, F167, L194, A305, E330, N330, A430, T431, I432, A476, I547, and/or K579.

18. The modified enzyme according to claim 17 comprising one or more amino acid substitution(s) selected from the group consisting of E7D, A33N, A153S, L159V, Y167F, I194L, T305A, D330E, D330N, S430A, S431T, V432I, I476A, V547I, R579K, and combinations thereof being introduced into a sequence with at least about 20%, such as 25, 30, 35, 40, 45, 50, 55, 60, 65, 70, 75, 80, 85, 90, 92, 95, 97, 98, 99% or up to 100% identity to SEQ ID NO:1 or shown in FIG. 1.

19. The modified enzyme according to claim 15 derived from Mucor circinelloides CarRP.

20. The modified enzyme according to claim 15, said enzyme being introduced and expressed in a suitable carotenoid and/or apocarotenoid-producing host cell, particularly retinoid-producing host cell.

21. The modified enzyme according to claim 15, wherein the catalytic activity towards production of apocarotenoids, particularly retinoids, is increased in the range of 20-65% or more, when expressed in a suitable apocarotenoid/retinoid-producing host cell as compared to the corresponding host cell expressing CarRP according to SEQ ID NO:1 or shown in FIG. 1.

22. The modified enzyme according to claim 15, wherein the catalytic activity towards production of carotenoids is increased in the range of 5-30%, when expressed in a suitable carotenoid-producing host cell as compared to the corresponding host cell expressing CarRP according to SEQ ID NO:1 or shown in FIG. 1.

23. A carotenoid and/or apocarotenoid producing host cell heterologous expressing the modified enzyme according to claim 15.

24. The host cell according to claim 23, which is a fungal host cell or selected from E. coli, preferably fungal host cell selected from Yarrowia or Saccharomyces.

25. The host cell according to claim 23 heterologous expressing genes involved in biosynthesis of a carotenoid and/or apocarotenoid selected from the group consisting of beta-carotene, lycopene, phytoene, beta-ionone, beta-cryptoxanthin, canthaxanthin, astaxanthin, zeaxanthin, rhodoxanthin, retinal, retinol, retinyl acetate, and mixtures thereof.

26. A process for production of carotenoids or apocarotenoids in a suitable host cell comprising:
(a) cultivating of a host cell according to claim 23 under suitable culture conditions expressing the modified enzyme,
(b) isolating and optionally purifying the carotenoids or apocarotenoids, from the cultivation medium,
wherein the percentage of carotenoids or apocarotenoidsis increased by at least about 5% as compared to a process using a host cell expressing CarRP according to SEQ ID NO: 1 or shown in FIG. 1 instead of said modified enzyme.

27. A process according to claim 26, wherein the carotenoids or aporacotenoids are selected from the group consisting of beta-carotene, lycopene, phytoene, beta-ionone, beta-cryptoxanthin, canthaxanthin, astaxanthin, zeaxanthin, rhodoxanthin, retinal, retinol, retinyl acetate, and mixtures thereof.

28. A method for increasing the productivity of an apocarotenoid producing host cell, particularly retinoid producing host cell, comprising:
(a) providing a host cell expressing genes involved in biosynthesis of apocarotenoids, particularly retinoids including but not limited to biosynthesis of retinal, retinol, and/or retinyl acetate;
(b) transforming said host cell with a polynucleotide expressing the modified enzyme according to claim 15,
(c) isolating and optionally purifying the apocarotenoids, particularly retinoids, including but not limited to retinal, retinol, and/or retinyl acetate from the host cell,
wherein the productivity is increased by at least about 20 to 75% as compared to a host cell of step (b) transformed with a polynucleotide according to SEQ ID NO:2 instead of a polynucleotide expressing said modified enzyme.
